(19) United States
(12) Patent Application Publication
(10) Pub. No.: US 2026/0088157 A1
(43) Pub. Date: Mar. 26, 2026

(54) Identifying Sets of Image Elements as Representative of a Sample Property for Pathology

(71) Applicant: Aiforia Technologies OYJ, Helsinki (FI)

(72) Inventors: Juha REUNANEN, Helsinki (FI); Kaisa HELMINEN, Helsinki (FI)

(21) Appl. No.: 19/341,468

(22) Filed: Sep. 26, 2025

Related U.S. Application Data
(63) Continuation of application No. PCT/FI2025/050479, filed on Sep. 17, 2025.
(60) Provisional application No. 63/699,623, filed on Sep. 26, 2024.

(51) Int. Cl. G16H 30/40

(52) U.S. Cl. CPC G16H 30/40

(57) ABSTRACT

A method of identifying sets of image elements as representative of a sample property for pathology includes receiving pathology image data representing sample images representing adjacent or overlapping portions of a sample for analysis in pathology, each of the sample images including sample image elements; causing a function to be applied to the sample images to determine confidence scores associated with the sample image elements and representing a level of confidence that the associated sample image element represents the sample property; comparing the confidence scores with a candidate confidence threshold to identify a candidate set of adjacent sample image elements, each associated with a confidence score greater than the candidate confidence threshold; determining whether a representative confidence score is greater than a confirmation confidence threshold; and, if so, associating the candidate set of adjacent sample image elements with a sample property identifier.

### RELATED APPLICATION

This application is a continuation of Patent Cooperation Treaty Application No. PCT/FI2025/050479 filed Sep. 17, 2025, which claims the benefit of U.S. Provisional Application No. 63/699,623 filed Sep. 26, 2024, the entire contents are hereby incorporated by reference in their entirety.

### BACKGROUND

### 1. Field

Embodiments of the present disclosure relate to image analysis for pathology and more particularly to identifying sets of image elements as representative of a sample property for pathology.

### 2. Description of Related Art

Image analysis in pathology may be used for identification of pathological properties in images. Some systems for identifying properties within an image may encounter misidentification including false negatives and/or false positives. Such systems may generally be tuned by adjusting threshold values. Increasing a threshold value may improve specificity but degrade sensitivity. Similarly, decreasing a threshold value may improve sensitivity while deteriorating specificity. Some traditional image analysis systems and/or methods may be unfeasible when applied to image analysis in pathology because image analysis in pathology may involve processing and/or manipulating pathology images, which in some cases may be extremely large, include multiple images, and/or be difficult to process and/or manipulate. For identification of pathological properties in pathology images, some known systems may be inaccurate, slow, costly, and/or inefficient.

### SUMMARY

In accordance with various embodiments, there is provided a method of identifying sets of image elements as representative of a sample property for pathology, the method including: receiving pathology image data representing a plurality of sample images representing respective adjacent or overlapping portions of a sample for analysis in pathology, each of the plurality of sample images including a plurality of sample image elements; causing one or more functions to be applied to the plurality of sample images to determine a plurality of confidence scores, each of the plurality of confidence scores associated with one of the plurality of sample image elements and representing a level of confidence that the associated sample image element represents the sample property; comparing at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold; determining whether at least one representative confidence score associated with the candidate set of adjacent sample image elements is greater than a confirmation confidence threshold; and, if the at least one representative confidence score is greater than the confirmation confidence threshold, associating the candidate set of adjacent sample image elements with a sample property identifier for identifying the candidate set of adjacent sample image elements as representing the sample property.

The method may include determining the at least one representative confidence score based on the confidence scores associated with the candidate set of adjacent sample image elements.

Determining the at least one representative confidence score may include identifying the at least one representative confidence score from the confidence scores associated with the candidate set of adjacent sample image elements.

The confirmation confidence threshold may be greater than the candidate confidence threshold.

The plurality of sample images may include a first sample image and a second sample image and comparing at least one of the plurality of confidence scores with the candidate confidence threshold to identify the candidate set of adjacent sample image elements may include: comparing confidence scores associated with sample image elements included in the first sample image with the candidate confidence threshold to identify a first sample image set of adjacent sample image elements, each sample image element of the first sample image set of adjacent sample image elements associated with a confidence score greater than the candidate confidence threshold; including the first sample image set of adjacent sample image elements in the candidate set of adjacent sample image elements; comparing confidence scores associated with sample image elements included in the second sample image with the candidate confidence threshold to identify a second sample image set of adjacent sample image elements, each sample image element of the second sample image set of adjacent sample image elements associated with a confidence score greater than the candidate confidence threshold; and determining whether at least one sample image element of the first sample image set is adjacent to or overlapping with at least one sample image element of the second sample image set, and, in response to determining that at least one sample image element of the first sample image set is adjacent to or overlapping with at least one sample image element of the second sample image set, including the second sample image set of adjacent sample image elements in the candidate set of adjacent sample image elements.

The at least one representative confidence score may include a representative high confidence score and determining whether the at least one representative confidence score is greater than the confirmation confidence threshold may include: determining the representative high confidence score based on the confidence scores associated with the candidate set of adjacent sample image elements; and determining whether the representative high confidence score is greater than the confirmation confidence threshold.

Determining the representative high confidence score may include: determining a first candidate representative high confidence score associated with a sample image element of the first sample image set; determining a second candidate representative high confidence score associated with a sample image element of the second sample image set; and determining the representative high confidence score as the greatest of the first and second candidate representative high confidence scores.

Associating the candidate set of adjacent sample image elements with the sample property identifier may include producing signals for causing the candidate set of adjacent sample image elements to be displayed in association with the sample property.

The method may include determining the plurality of sample images based on the received pathology image data.

Determining the plurality of sample images based on the received pathology image data may include: determining a candidate set of sample images; identifying at least one undesirable sample image of the candidate set of sample images; and determining the plurality of sample images as a subset of the candidate set of sample images, the plurality of sample images not including the at least one undesirable sample image.

Identifying the at least one undesirable sample image may include determining that the at least one undesirable sample image generally lacks depiction of tissue.

Identifying the at least one undesirable sample image may include causing sample image elements of the candidate set of sample images to be input into an undesirable image element detecting neural network.

The pathology image data may include a representation of a single pathology image and determining the plurality of sample images based on the received pathology image data may include determining the plurality of sample images as portions of the pathology image.

Determining the plurality of sample images from the pathology image data may include, for each of the plurality of sample images: determining a position of the sample image within the pathology image and associating the position with the sample image; and associating a width and a height for the sample image with the sample image.

The plurality of sample images may be overlapping by an overlap width and an overlap height between adjacent sample images.

Causing the one or more functions to be applied to the plurality of sample images to determine a plurality of confidence scores may include causing each of the plurality of sample images to be input into a property identifying neural network.

Causing the one or more functions to be applied to the plurality of sample images to determine a plurality of confidence scores may include causing each of the plurality of sample images to be input into a property identifying neural network, the property identifying neural network having a limited field of view having a field of view width and a field of view height; the plurality of sample images may be overlapping by an overlap width and an overlap height between adjacent sample images; and the overlap height may be greater than or equal to the field of view height minus one image element height and the overlap width is greater than or equal to the field of view width minus one image element width.

The overlap width may be equal to the field of view width minus one image element width and the overlap height may be equal to the field of view height minus one image element height.

The candidate set of adjacent sample image elements may include sample image elements from more than one of the plurality of sample images.

The sample property may include at least one of: a biomarker; a type of tissue; epithelial tissue; stroma; neither epithelial tissue nor stroma; properties of a cell; nuclei; cell membrane; mitotic status; cells expressing a specific protein; cells expressing Ki-67; a cell or group of cells having a condition; a cancer cell; a group of cancer cells forming a tumor; an immune cell; a cell having a pathological condition; a necrotic cell; a histologic pattern; a Gleason pattern; or a tumor grade.

The sample image element may be a pixel.

In accordance with various embodiments, there is provided a system for identifying sets of image elements as representative of a sample property for pathology, the system comprising at least one processor configured to perform any of the above methods.

In accordance with various embodiments, there is provided a non-transitory computer-readable medium having stored thereon codes that when executed by at least one processor cause the at least one processor to perform any of the above methods.

In accordance with various embodiments, there is provided a system for identifying sets of image elements as representative of a sample property for pathology, the system including: provisions for receiving pathology image data representing a plurality of sample images representing respective adjacent or overlapping portions of a sample for analysis in pathology, each of the plurality of sample images including a plurality of sample image elements; provisions for causing one or more functions to be applied to the plurality of sample images to determine a plurality of confidence scores, each of the plurality of confidence scores associated with one of the plurality of sample image elements and representing a level of confidence that the associated sample image element represents the sample property; provisions for comparing at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold; provisions for determining whether at least one representative confidence score associated with the candidate set of adjacent sample image elements is greater than a confirmation confidence threshold; and provisions for, if the at least one representative confidence score is greater than the confirmation confidence threshold, associating the candidate set of adjacent sample image elements with a sample property identifier for identifying the candidate set of adjacent sample image elements as representing the sample property.

Other aspects and features of embodiments of the present disclosure will become apparent to those ordinarily skilled in the art upon review of the following description of specific embodiments of the present disclosure in conjunction with the accompanying figures.

### BRIEF DESCRIPTION OF THE DRAWINGS

In drawings which illustrate embodiments of the present disclosure,

FIG. 1 is a schematic view of a system for identifying sets of image elements as representative of a sample property for pathology, according to various embodiments;

FIG. 2 is a representation of a pathology image represented by pathology image data that may be used in the system shown in FIG. 1, in accordance with various embodiments;

FIG. 3 is a representation of a plurality of sample images represented by the pathology image data that may be used in the system shown in FIG. 1, in accordance with various embodiments;

FIG. 4 is a representation of a portion of the pathology image shown in FIGS. 2 and 3 shown enlarged and in further detail with sample images delineated, in accordance with various embodiments;

FIG. 5 is a schematic view of an image analyzer of the system shown in FIG. 1, including a processor circuit, in accordance with various embodiments;

FIG. 6 is a flowchart depicting blocks of code for directing the image analyzer shown in FIG. 5 to perform identifying sets of image elements as representative of a sample property for pathology, in accordance with various embodiments;

FIG. 7 is a flowchart depicting blocks of code that may be included in the flowchart shown in FIG. 6, in accordance with various embodiments;

FIG. 8 is a representation of a sample image property record that may be used in the system shown in FIG. 1, in accordance with various embodiments;

FIG. 9 is a representation of a candidate sample image definition record that may be used in the system shown in FIG. 1, in accordance with various embodiments;

FIG. 10 is a representation of a sample image definition record that may be used in the system shown in FIG. 1, in accordance with various embodiments;

FIG. 11 is a representation of a property confidence score record that may be used in the system shown in FIG. 1, in accordance with various embodiments;

FIG. 12 is a flowchart depicting blocks of code that may be included in the flowchart shown in FIG. 6, in accordance with various embodiments;

FIG. 13 is a representation of a candidate confidence threshold comparison record that may be used in the system shown in FIG. 1, in accordance with various embodiments;

FIG. 14 is a representation of a candidate confidence threshold comparison contour record that may be used in the system shown in FIG. 1, in accordance with various embodiments;

FIG. 15 is a representation of a grouped candidate set identifier record that may be used in the system shown in FIG. 1, in accordance with various embodiments;

FIG. 16 is a representation of a candidate confidence threshold comparison contour record that may be used in the system shown in FIG. 1, in accordance with various embodiments;

FIG. 17 is a representation of an updated version of the grouped candidate set identifier record shown in FIG. 15, in accordance with various embodiments;

FIG. 18 is a flowchart depicting blocks of code that may be included in the flowchart shown in FIG. 6, in accordance with various embodiments;

FIG. 19 is a flowchart depicting blocks of code that may be included in the flowchart shown in FIG. 18, in accordance with various embodiments;

FIG. 20 is a representation of a grouped candidate set representative scores record that may be used in the system shown in FIG. 1, in accordance with various embodiments;

FIG. 21 is a representation of an updated version of the grouped candidate set representative scores record shown in FIG. 20, in accordance with various embodiments;

FIG. 22 is a representation of an updated version of the grouped candidate set representative scores record shown in FIG. 20, in accordance with various embodiments;

FIG. 23 is a representation of a grouped confirmed set identifier record that may be used in the system shown in FIG. 1, in accordance with various embodiments;

FIG. 24 is a depiction of a block of code that may be included in the flowchart shown in FIG. 6, in accordance with various embodiments;

FIG. 25 is a representation of a set of adjacent sample image elements displayed in association with a sample property that may be displayed by a display of the system shown in FIG. 1, in accordance with various embodiments; and

FIG. 26 is a flowchart depicting blocks of code that may be included in the flowchart shown in FIG. 6, in accordance with various embodiments.

### DETAILED DESCRIPTION

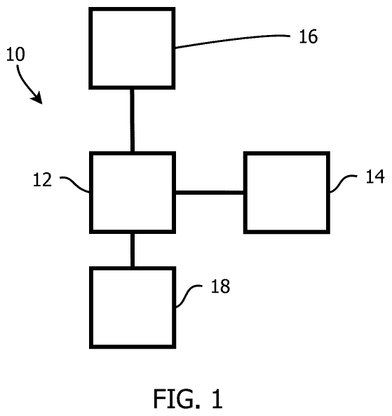

Referring to FIG. 1, there is provided a system 10 for identifying sets of image elements as representative of a sample property for pathology in accordance with various embodiments. The system 10 includes an image analyzer 12 in communication with an image source 14. In some embodiments, the system includes a display 16 in communication with the image analyzer 12. In some embodiments, the system 10 may include a Laboratory Information System (LIS) 18 in communication with the image analyzer 12. In various embodiments, the system 10 may facilitate accurate, fast, low cost, and/or efficient analysis of pathology images.

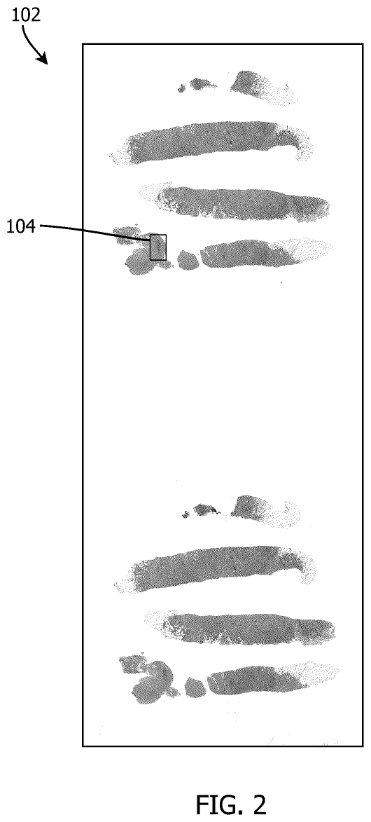

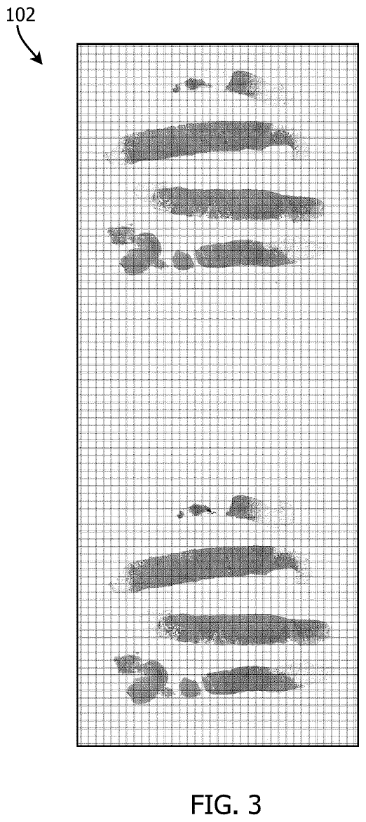

In various embodiments, the image analyzer 12 may be configured to receive pathology image data representing a plurality of sample images or sub images. Referring to FIG. 2, there is shown a pathology image 102 represented by the pathology image data received by the image analyzer 12 in accordance with various embodiments. In various embodiments, the pathology image data may represent a large complete image and the image analyzer 12 may be configured to determine or define a plurality of sample images based on the pathology image data. For example, in some embodiments, the image analyzer 12 may be configured to define sample images or sub images from the pathology image 102, which may be generally tiled and/or overlapping on the pathology image 102, for example, as shown in FIG. 3.

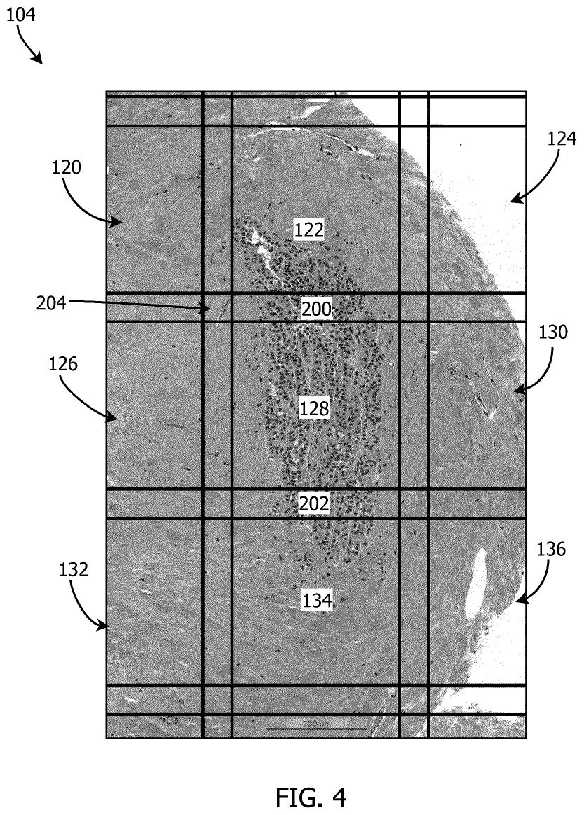

Referring to FIG. 4, a portion 104 of the complete pathology image 102 shown in FIG. 2 is shown enlarged and in further detail, with sample images delineated, for illustrative purposes. In various embodiments, the plurality of sample images shown in FIG. 3 may include sample images or sub images 120, 122, 124, 126, 128, 130, 132, 134, and 136 of the portion 104 shown in FIG. 4. Notably, in FIG. 4, the entirety of sample images 122, 128, 134 are shown and portions of sample images 120, 124, 126, 130, 132, and 136 are shown. Borders of the sample images 120-136 are shown in dark lines. In various embodiments, the sample images may include images for analysis in pathology or pathology images and/or may together represent the single pathology image 102. In some embodiments, the sample images may represent a hematoxylin and eosin stain, for example.

In various embodiments, the sample images including the images 120-136 may represent respective partially overlapping images or portions of a sample for analysis in pathology. For example, borders of the sample images 120-136 are shown in FIG. 4, showing overlapping portions, including for example, overlapping portion 200, which is common between sample images 122 and 128, overlapping portion 202, which is common between sample images 128 and 134, and overlapping portion 204, which is common between sample images 120, 122, 126, and 128. In some embodiments, the sample images 120-136 may be at least partially overlapping. In some embodiments overlapping sample images 120-136 may facilitate analysis of the pathology image data while reducing or avoiding gaps, incomplete analysis, and/or discontinuities.

In various embodiments, the use of a plurality of sample images that represent respective overlapping or adjacent portions of a sample for analysis in pathology may facilitate manipulation and/or processing of large quantities/extents of image data representing the sample while also facilitating accurate machine analysis of the image data.

In some embodiments, sample images may be defined such that the sample images are overlapping by an overlap width and an overlap height between adjacent sample images. In some embodiments, the overlap width and the overlap height may be chosen based on a property identifying neural network's (described in further detail herein) limited field of view, so that transitions between adjacent sample images are smooth and/or context for features in the overlapping portions is properly considered by the neural network during analysis. In some embodiments, the extent of the overlapping portions may be chosen to reduce or minimize redundant processing.

In some embodiments, sample images may need to be small enough so that each of them will fit in GPU memory, together with the processing results and any intermediate and/or other data that may also need to be stored in GPU memory before, during, and/or after processing. As long as the sample images do fit in the available GPU memory it may be desirable to maximize the sample image size so that the total amount of overlap is reduced, which may speed up processing. In various embodiments, for example, the plurality of sample images may together represent an image having 30,000 megapixels of image data, which may be unfeasible for storing and/or processing in GPU memory at any one time, and each sample image may represent 3 megapixels of image data, for example. In various embodiments, the plurality of sample images may together represent a whole-slide image (WSI) used in pathology.

In some embodiments, the image analyzer 12 may receive the pathology image data from the image source 14. In various embodiments, the image source 14 may have previously received and stored the pathology image data for analysis. In some embodiments, the image source 14 may include a picture archiving and communication system (PACS), for example. In some embodiments, the image analyzer 12 may be configured to provide regular PACS functionality and the image source 14 may include a slide scanner, for example.

In some embodiments, the sample images including the sample images 120-136 shown in FIG. 4 may represent a microscope image of patient tissue that is to be analyzed, such as by machine and/or human analysis for pathology. In various embodiments, the image analyzer 12 may be configured to analyze the pathology image data to identify sets of image elements included in the image data as representative of a sample property for pathology. For example, in some embodiments, the image analyzer 12 may be configured to analyze the pathology image data to identify or categorize sets or groups of adjacent pixels in the sample images as representative of one or more of the following objects or properties: a biomarker, a type of tissue, such as, for example, epithelial tissue, stroma, or neither, and/or properties of the cell, such as: nuclei, cell membrane, mitotic status, or cells expressing a specific protein, such as, for example, Ki-67; a cell or group of cells having a condition, such as, for example, a cancer cell or group of cancer cells forming a tumor, an immune cell, or a cell having another pathological condition (e.g., a necrotic cell); a histologic pattern, such as a Gleason pattern; a tumor grade; or another property. In various embodiments, this identification may be useful in pathology, for example, for further diagnostics of the identified tissue and/or in deriving statistics or metrics associated with the identified tissue. In some embodiments, the image analyzer 12 may be configured to produce signals based on the analysis. For example, in some embodiments, the image analyzer 12 may be configured to produce signals for causing the sets of pixels to be displayed in association with the property or for causing further action to be taken, such as, for example, having additional stains be ordered. In some embodiments, the image analyzer 12 may be configured to derive or determine metrics related to the sets of pixels, such as total area, for example, and the image analyzer 12 may be configured to produce signals for causing the metrics to be provided to another system and/or displayed to a user (e.g., a pathologist).

In various embodiments, the image analyzer 12 may be configured to cause one or more functions to be applied to the plurality of sample images including the sample images 120-136 to determine a plurality of confidence scores, each of the plurality of confidence scores associated with one of the plurality of sample image elements and representing a level of confidence that the associated sample image element represents the sample property. In various embodiments, the sample image elements may be pixels. Accordingly, in some embodiments, the image analyzer 12 may be configured to apply a neural network function to each of the sample images including the sample images 120-136 to determine a plurality of confidence scores, each of the plurality of confidence scores associated with a pixel and representing a level of confidence that the associated pixel represents a property such as, for example, those listed above. In some embodiments, the neural network function may be a fully convolutional network and the image analyzer 12 may be configured to apply to each of the sample images the fully convolutional network, such as, as described in Fully Convolutional Networks for Semantic Segmentation; Long, J., Shelhamer, E., Darrell, T. (2014) (https://arxiv.org/abs/1411.4038). In various embodiments, the image analyzer 12 may be configured to apply a U-Net, such as, as described in U-Net: Convolutional Networks for Biomedical Image Segmentation; Ronneberger, O., Fischer, P., Brox, T. (2015) (https://arxiv.org/abs/1505.04597). In some embodiments, the image analyzer 12 may be configured to apply a vision transformer, such as, as described in An Image is Worth 16×16 Words: Transformers for Image Recognition at Scale; Dosovitskiy, A., et al. (2021) (https://arxiv.org/abs/2010.11929) and/or its various follow-ups.

In some embodiments, confidence scores resulting from neural network analysis for each of the pixels in overlapping portions or zones of the sample images may be selected from the results of neural network analysis for sample image(s) where the relevant field of view is defined for each of the pixels. In various embodiments, using a convolutional neural network or a fully convolutional neural network may facilitate having a limited field of view parameter, and therefore having a well-defined context for each pixel in at least one of the overlapping sample images.

In various embodiments, confidence scores resulting from neural network analysis of the pixels in the overlapping portions may be combined. In some embodiments, confidence scores may be bilinearly interpolated, such that each individual sample image result contributes relatively more whenever the target pixel is relatively farther away from the edge of the corresponding sample image; for example, when combining confidence scores in the overlapping portion 200 common between sample images 122 and 128 shown in FIG. 4, confidence scores associated with the pixels in a top part of the overlapping portion 200 nearest a center of the sample image 122 may be based mainly on neural network processing of the sample image 122, whereas pixels in a bottom part of the overlapping portion 200 nearest a center of the sample image 128 may be based mainly on neural network processing of the sample image 128. Similarly, in the corner overlapping portion 204 common between 120, 122, 126, and 128, pixels in the top left corner nearest a center of the sample image 120 may be based mainly on neural network processing of the sample image 120. In various embodiments, combining confidence scores in the overlapping portions based on individual sample image results may be particularly useful whenever the neural network is not a straightforward convolutional net having a limited field of view (but instead is a vision transformer, for example).

In various embodiments, the image analyzer 12 may be configured to use a candidate confidence threshold and a confirmation confidence threshold for comparison with confidence scores. In some embodiments, the candidate confidence threshold and the confirmation confidence threshold may be compared to the same group of confidence scores and the confirmation confidence threshold may be greater than the candidate confidence threshold and so the confirmation confidence threshold may act as a high confidence threshold and the candidate confidence threshold may act as a low confidence threshold. In various embodiments use of both the candidate confidence and confirmation confidence thresholds may facilitate improved accuracy in determining whether a set of image elements is representative of a sample property for pathology. In some embodiments, use of both the candidate confidence and confirmation confidence thresholds may facilitate an increase in specificity with reduced sacrifice of sensitivity of the analysis, may facilitate an increase in sensitivity with reduced sacrifice of specificity, and/or may improve both specificity and sensitivity.

In some embodiments, the image analyzer 12 may be configured to compare at least one of the plurality of confidence scores with the candidate confidence threshold to identify a candidate set of adjacent sample image elements of the plurality of sample image elements, each sample image element of the candidate set of adjacent sample image elements associated with a confidence score greater than the candidate confidence threshold. In some embodiments, the image analyzer 12 may be configured to include or group sample image elements from more than one of the sample images into the candidate set of adjacent sample image elements. In various embodiments, by grouping sample image elements from more than one of the sample images into the candidate set of adjacent sample image elements, analysis can be improved in applications with large size images, such as, for example, in pathological image analysis.

In various embodiments, each pixel associated with a confidence score that is greater than the candidate confidence threshold may be included in a respective group or set of adjacent pixels that are associated with a confidence score that is greater than the candidate confidence threshold. In some embodiments, the group or set of adjacent pixels may be considered as a candidate set of pixels that could be determined as representative of a sample property, but only if a further condition is met. For example, in some embodiments, the further condition may require that at least one of the pixels in the candidate set of pixels is associated with a confidence score that is greater than a confirmation confidence threshold. In some embodiments, the confirmation confidence threshold may be greater than the candidate confidence threshold. In various embodiments, requiring that at least one of the pixels in the candidate set of pixels be associated with a confidence score that is greater than the confirmation confidence threshold may facilitate an increase in specificity with reduced sacrifice of sensitivity of the analysis.

In various embodiments, the image analyzer 12 may be configured to determine whether a representative confidence score associated with the candidate set of adjacent sample image elements is greater than a confirmation confidence threshold. In some embodiments, the representative confidence score may be determined based on the confidence scores associated with the candidate set of adjacent sample image elements. However, in other embodiments, the representative confidence score may be determined separately from the confidence scores associated with the candidate set of adjacent sample image elements.

In some embodiments, such as, for example where the representative confidence score is determined based on the confidence scores associated with the candidate set of adjacent sample image elements, the confirmation confidence threshold may be greater than the candidate confidence threshold. In some embodiments, the representative confidence score may be determined by identifying the representative confidence score from the confidence scores associated with the candidate set of adjacent sample image elements. For example, in some embodiments, the representative confidence score may be chosen as a confidence score associated with a pixel of the candidate set of adjacent pixels. In some embodiments, the image analyzer 12 may be configured to determine a highest confidence score and to use the highest confidence score as the representative confidence score. In various embodiments, using a highest confidence score as the representative confidence score may facilitate fast or efficient processing while maintaining improved specificity and sensitivity. In some embodiments, other representative confidence scores may be used, such as, for example, an Nth highest confidence score, wherein N is a number greater than 1. In various embodiments, the Nth highest confidence score may be a score above which there are (N−1) pixels having higher confidence scores. In some embodiments, the value of N may be 3, for example. In various embodiments, using the Nth highest confidence score, wherein N is greater than 1 may facilitate avoiding or reducing the likelihood that a single outlier or noisy pixel value may cause a false confirmation.

In various embodiments, the image analyzer 12 may be configured to, if the at least one representative confidence score is greater than the confirmation confidence threshold, associate the candidate set of adjacent sample image elements with a sample property identifier for identifying the candidate set of adjacent sample image elements as representing the sample property. For example, in some embodiments, the image analyzer 12 may be configured to determine whether the highest confidence score is greater than the confirmation confidence threshold and, if the highest confidence score is greater than the confirmation confidence threshold, determine that the candidate set of adjacent sample image elements represents the sample property.

For example, in some embodiments, the image analyzer 12 may be configured to associate the candidate set of adjacent sample image elements with a sample property identifier by producing signals for causing the candidate set of adjacent sample image elements to be displayed in association with the sample property, such as, for example by being displayed and/or identified or highlighted on the display 16.

In various embodiments, the image analyzer 12 may be configured to associate the candidate set of adjacent sample image elements with a sample property identifier by producing signals for causing action to be taken. For example, in some embodiments, the image analyzer 12 may be configured to produce signals for causing at least one new sample staining to be ordered from a laboratory. In some embodiments, if a tumor has been detected, some additional biomarker stainings may be ordered automatically from the sample handling laboratory. In some embodiments, the image analyzer 12 may be configured to transmit a message to the LIS 18, the message including a patient ID, sample ID, stain type, an indication that the patient has a cancerous tumor, and/or an area of the tumor. In various embodiments, the LIS 18 may be configured to receive the message and cause at least one additional biomarker staining to be ordered based on the message.

In some embodiments, the image analyzer 12 may be configured to derive or determine metrics related to the candidate set of adjacent sample image elements, such as total area and/or grade of tumor, for example, and the image analyzer 12 may be configured to produce signals for causing the metrics to be displayed to a user (e.g., a pathologist). In some embodiments, the image analyzer 12 may be configured to input the candidate set of adjacent sample image elements and/or additional contextual image data into a tumor grade determining neural network trained to output a detection and/or classification result representing a tumor grade for the candidate set of adjacent sample image elements. In various embodiments, the tumor grade determining neural network may be trained, i.e. first manually annotating each tumor (or part of tumor) in the training data accordingly (e.g., as a “grade 3” or “grade 4” or “grade 5” tumor), then training the tumor grade determining neural network.

In some embodiments, the metrics may be included in a report and the report may be sent to a pathologist and/or to an oncologist for review. In some embodiments, the metrics may affect treatment decisions (e.g., surgery, chemotherapy, radiation, and/or another treatment decision). In some embodiments, the image analyzer 12 may be configured to produce signals for causing automated treatment recommendation and/or decision to be performed. In various embodiments, a human expert may review and manually override the automated treatment decision.

In various embodiments, the image analyzer 12 may be configured to use the determined metrics to automatically prioritize treatment for patients. In some embodiments, a relatively higher-grade tumor area may lead to a patient case receiving a relatively higher priority, for example. In various embodiments, a human expert may review and manually override the automatic prioritization of patient cases. In some embodiments, the image analyzer 12 may be configured to produce signals for causing a worklist to be generated and/or displayed to a user, wherein the worklist includes patient cases listed or ordered in a prioritized order based on one or more of the determined metrics (e.g., ordered such that patient cases having higher grade tumors are listed or ranked first).

In various embodiments, the worklist may be generated based on availability and/or expertise of candidate sample reviewing experts, such as pathologists. For example, in some embodiments, the image analyzer 12 may be configured to produce signals for causing relatively difficult patient cases to be directed to relatively experienced sample reviewing experts, and relatively easy patient cases to be directed to relatively inexperienced sample reviewing experts. For example, in some embodiments, borderline cases where the accuracy of the image analysis results may critically affect the patient being eligible/non-eligible for a specific treatment may be considered as relatively difficult patient cases and may be directed to relatively more experienced sample reviewing experts, and some clearly positive or negative cases may be directed to any expert.

### Image Analyzer—Processor Circuit

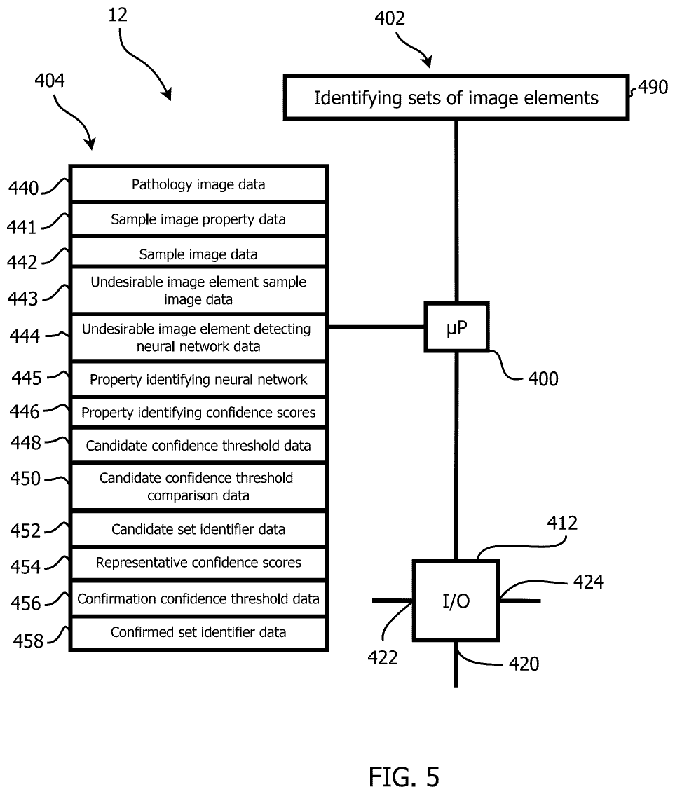

Referring now to FIG. 5, a schematic view of the image analyzer 12 of the system 10 shown in FIG. 1 according to various embodiments is shown. Referring to FIG. 5, the image analyzer 12 includes a processor circuit including an analyzer processor 400 and a program memory 402, a storage memory 404, and an input/output (I/O) interface 412, all of which are in communication with the analyzer processor 400. In various embodiments, the analyzer processor 400 may include one or more processing units, such as for example, a central processing unit (CPU), a graphics processing unit (GPU), and/or a field programmable gate array (FPGA). In some embodiments, any or all of the functionality of the image analyzer 12 described herein may be implemented using one or more FPGAs.

The I/O interface 412 includes an interface 420 for communicating with the image source 14 shown in FIG. 1. In some embodiments, the I/O interface 412 includes an interface 422 for communicating with the display 16 shown in FIG. 1. In some embodiments, the I/O interface 412 may include an interface 424 for communicating with the LIS 18. In some embodiments, the I/O interface 412 may include an interface for facilitating networked communication through a network such as the Internet and/or one or more interfaces for enabling user input via one or more user interface devices, such as, for example, a pointer and/or a keyboard. In some embodiments, any or all of the interfaces may facilitate wireless and/or wired communication. In some embodiments, each of the interfaces may include one or more interfaces and/or some or all of the interfaces may be implemented as combined interfaces or a single interface.

In some embodiments, where a device is described herein as receiving or sending information, it may be understood that the device receives signals representing the information via an interface of the device or produces signals representing the information and transmits the signals to the other device via an interface of the device.

Processor-executable program codes for directing the analyzer processor 400 to carry out various functions are stored in the program memory 402. Referring to FIG. 5, the program memory 402 includes a block of codes 490 for directing the image analyzer 12 to perform identifying sets of image elements as representative of a sample property for pathology functions. In this specification, it may be stated that certain encoded entities such as applications or modules perform certain functions. Herein, when an application, module or encoded entity is described as taking an action, as part of, for example, a function or a method, it will be understood that at least one processor (e.g., the analyzer processor 400) is directed to take the action by way of programmable codes or processor-executable codes or instructions defining or forming part of the application.

The storage memory 404 includes a plurality of storage locations including location 440 for storing pathology image data, location 441 for storing sample image or sub image property data, location 442 for storing sample image or sub image data, location 443 for storing undesirable image element sample image data, location 444 for storing undesirable image element detecting neural network data, location 445 for storing property identifying neural network data, location 446 for storing property identifying confidence score data, location 448 for storing candidate confidence threshold data, location 450 for storing candidate confidence threshold comparison data, location 452 for storing candidate set identifier data, location 454 for storing representative confidence score data, location 456 for storing confirmation confidence threshold data, and location 458 for storing confirmed set identifier data. In various embodiments, the plurality of storage locations may be stored in a database in the storage memory 404.

In various embodiments, the block of codes 490 may be integrated into a single block of codes or portions of the block of codes 490 may include one or more blocks of code stored in one or more separate locations in the program memory 402. In various embodiments, any or all of the locations 440-458 may be integrated and/or each may include one or more separate locations in the storage memory 404.

Each of the program memory 402 and storage memory 404 may be implemented as one or more storage devices including random access memory (RAM), a hard disk drive (HDD), a solid-state drive (SSD), a network drive, flash memory, a memory stick or card, any other form of non-transitory computer-readable memory or storage medium, and/or a combination thereof. In some embodiments, the program memory 402, the storage memory 404, and/or any portion thereof may be included in a device separate from the image analyzer 12 and in communication with the image analyzer 12 via the I/O interface 412, for example. In some embodiments, the functionality of the analyzer processor 400 and/or the image analyzer 12 as described herein may be implemented using a plurality of processors and/or a plurality of devices.

### Operation

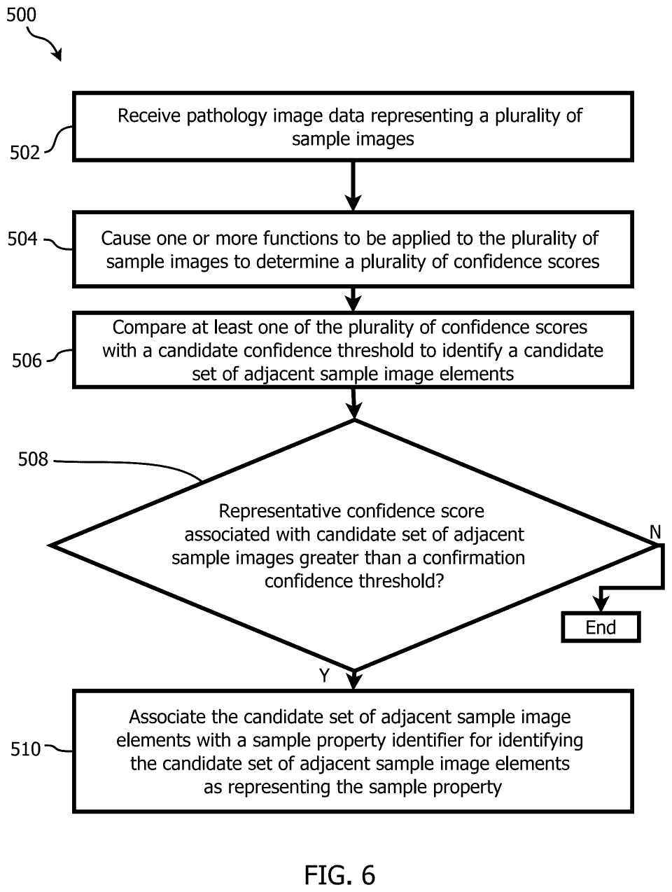

As discussed above, in various embodiments, the image analyzer 12 shown in FIGS. 1 and 5 may be configured to identify sets of image elements as representative of a sample property for pathology. Referring to FIG. 6, a flowchart depicting blocks of code for directing the analyzer processor 400 shown in FIG. 5 to perform identifying sets of image elements as representative of a sample property for pathology in accordance with various embodiments is shown generally at 500. In various embodiments, the blocks of code included in the flowchart 500 may be encoded in the block of codes 490 of the program memory 402 shown in FIG. 5.

In some embodiments, the flowchart 500 may be executed to perform or facilitate identification of groups or sets of pixels in pathology sample images as representative of one or more of the following objects or properties: a biomarker, a type of tissue, such as, for example, epithelial tissue, stroma, or neither, and/or properties of the cell, such as: nuclei, cell membrane, mitotic status, or cells expressing a specific protein, such as, for example, Ki-67; a cell or group of cells having a condition, such as, for example, a cancer cell or group of cancer cells forming a tumor, an immune cell, or a cell having another pathological condition (e.g., a necrotic cell); a histologic pattern, such as a Gleason pattern; a tumor grade; or another property.

Referring to FIG. 6, the flowchart 500 begins with block 502 which directs the analyzer processor 400 to receive pathology image data representing a plurality of sample images representing respective adjacent or overlapping portions of a sample for analysis in pathology, each of the plurality of sample images including a plurality of sample image elements.

In some embodiments, block 502 of the flowchart 500 shown in FIG. 6 may direct the analyzer processor 400 shown in FIG. 5 to receive pathology image data representing the pathology image 102 shown in FIG. 2.

In some embodiments, block 502 may direct the analyzer processor 400 to receive the pathology image data representing the pathology image 102 shown in FIG. 2 from the image source 14 shown in FIG. 1 via the interface 420 of the I/O interface 412 shown in FIG. 5, for example. In various embodiments, the pathology image 102 may include a plurality of pixels, each pixel including at least one pixel value associated with a position in the pathology image 102. For example, in some embodiments, the pathology image 102 may be a color image and each pixel may include three separate color components, which may include red, green and blue (RGB) pixel values, or hue, saturation and intensity (HSI) pixel values associated with a position in the pathology image 102. In various embodiments, each of the pixel values may be an 8-bit integer value from 0 to 255. In some embodiments, each of the pixel values may be a 16-bit integer value from 0 to 65535. In some embodiments, each of the pixel values may be represented as a floating-point number, such as the 32-bit single-precision format defined by the IEEE Standard for Floating-Point Arithmetic (IEEE 754).

In some embodiments, block 502 of the flowchart 500 shown in FIG. 6 may include code for directing the analyzer processor 400 to determine or identify the plurality of sample images based on the received pathology image data. In various embodiments, breaking up or dividing the pathology image 102 by determining the sample images based on the received pathology image data rather than analyzing the entire pathology image 102 undivided, may facilitate manipulation or processing of large data sets included in the pathology image data.

In some embodiments, determining the plurality of sample images based on the received pathology image data may involve determining the plurality of sample images as portions of the pathology image 102. In various embodiments, breaking up or dividing the pathology image 102 into smaller sample images may facilitate manipulation or processing of a large image, which may be commonly used in pathology, for example.

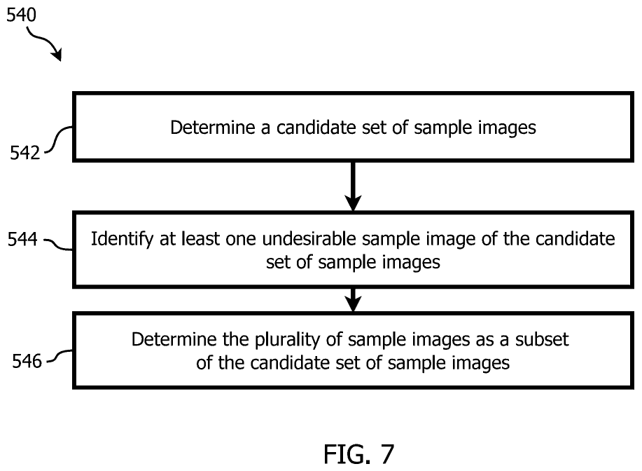

Referring to FIG. 7, there is shown a flowchart 540 depicting blocks of code that may be included in the block 502 in accordance with various embodiments. In various embodiments, the flowchart 540 may direct the analyzer processor 400 to determine the plurality of sample images based on the received pathology image data.

The flowchart 540 begins with block 542, which directs the analyzer processor 400 to determine a candidate set of sample images. In some embodiments, block 542 may direct the analyzer processor 400 to read from the location 441 of the storage memory 404 shown in FIG. 5, a height and a width to be used for the sample images. In some embodiments, block 542 may direct the analyzer processor 400 to read from the location 441 of the storage memory 404 shown in FIG. 5, an overlap height and an overlap width to be used for the sample images.

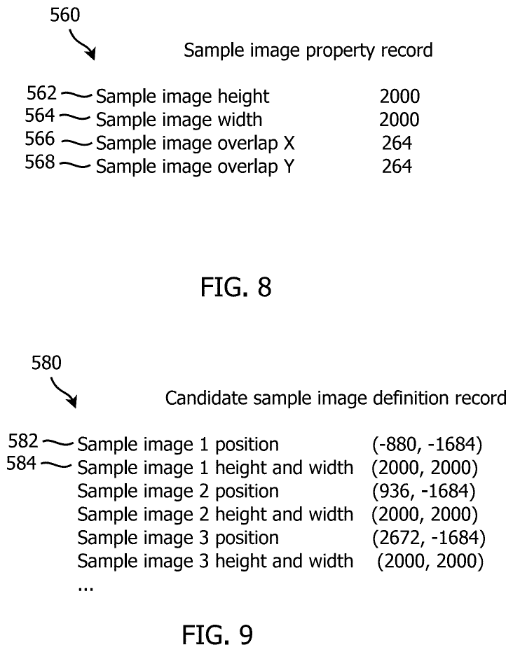

In some embodiments, a sample image property record 560 as shown in FIG. 8 may have been previously defined and stored in the location 441 of the storage memory 404, the sample image property record 560 including a sample image height field 562 for storing a sample image height in pixels and a sample image width field 564 for storing a sample image width in pixels. The sample image property record 560 may also include a sample image overlap X field 566 for storing a sample image overlap width in the X direction in pixels and a sample image overlap Y field 568 for storing a sample image overlap height in the Y direction in pixels.

In some embodiments, the X and Y overlaps for the sample images stored in the sample image property record 560 may be 264 pixels and 264 pixels respectively (63.9 micrometers, for example). In some embodiments, the Y-direction height and X-direction width for the sample images stored in the sample image property record 560 may be 2000 pixels and 2000 pixels respectively, for example.

In various embodiments, block 542 may direct the analyzer processor 400 to identify or define candidate sample images within the pathology image 102 such that the pathology image 102 is tiled or covered by the candidate sample images, each of the sample images having a height and width as read from the sample image property record 560 as shown in FIG. 8 and having an overlap height and width as read from the sample image property record 560. In some embodiments, block 542 may direct the analyzer processor 400 to start tiling from the center of the pathology image 102, such that the center of the pathology image 102 coincides with the center of a sample image of the candidate set of sample images. In some embodiments, if there would be fewer sample images required by making the center of the image be between two sample images (and not in the center of a sample image), then block 542 may direct the analyzer processor 400 to do that instead. In various embodiments, this decision may be made separately for the X and the Y dimensions.

In some embodiments, block 542 may direct the analyzer processor 400 to, for each of the candidate set of sample images: determine a position of the sample image within the pathology image 102 and associate the position with the sample image. In various embodiments, block 542 may direct the analyzer processor 400 to associate a width and a height for the sample image with the sample image. In some embodiments, the sample images may be defined by a candidate sample image definition record 580 as shown in FIG. 9. In some embodiments, block 542 may direct the analyzer processor 400 to determine and store in the location 442 of the storage memory 404 shown in FIG. 5 the candidate sample image definition record 580 as shown in FIG. 9. Referring to FIG. 9, the candidate sample image definition record 580 includes a first sample image position field 582 for storing a pixel based position of a first sample image taken from within the pathology image 102 and a first sample image height and width field 584 for storing a pixel based height and width of the first sample image.

In an illustrative embodiment, the sample image defined by the first sample image position field 582 and the first sample image height and width field 584 may be an upper left most sample image and block 542 may direct the analyzer processor 400 to set the first sample image position field 582 such that the top left corner of the first sample image is at an (X, Y) pixel position of (−880, −1684). In some embodiments, this may facilitate having the sample images be centered with respect to the pathology image 102. In various embodiments, having the sample images be centered may lead to treating the edges and/or corners of the pathology image 102 similarly (relative to each other), without making the top left corner be a special case, for example. In some embodiments, this may improve analysis accuracy and/or consistency.

In various embodiments, block 542 may direct the analyzer processor 400 to generate and store respective sample image position fields and sample image height and width fields to cover the entire pathology image 102 shown in FIG. 2 such that the defined candidate sample images are overlapping by an overlap width and an overlap height between adjacent sample images corresponding to those defined by the sample image property record 560 shown in FIG. 8. In some embodiments, the candidate sample image definition record 580 may include a single height and a single width field, which is associated with and applies to all of the sample images instead of having separate height and width fields for each of the sample images.

In various embodiments, each of the candidate sample images including respective pixels may be determined using the candidate sample image definition record 580 shown in FIG. 9 and the pathology image 102 shown in FIG. 2 included in the received pathology image data. In various embodiments, for each of the candidate sample images, an associated image position or offset for the sample image may be stored, such that a global pixel position relative to other sample images and/or within the pathology image 102 may be determined for each pixel position in a respective sample image.

Referring to FIG. 7, block 544 directs the analyzer processor 400 to identify at least one undesirable sample image of the candidate set of sample images. In some embodiments, identifying and then not considering undesirable sample images may facilitate improved processing and/or reduced wasted effort in processing, for example. In some embodiments, at block 544, no undesirable sample images may be identified and block 544 may direct the analyzer processor 400 to proceed to block 546 with no undesirable sample images identified. However, in some embodiments, at block 544, at least one undesirable sample images may be identified.

In some embodiments, block 544 may direct the analyzer processor 400 to determine that the at least one undesirable sample image generally lacks depiction of tissue. In various embodiments, the pathology image 102 may include regions where no tissue is shown and reviewing or analyzing those regions in detail (such as required for tumor detection and/or grading, for example) may be wasted effort. Accordingly, in some embodiments, identifying and ignoring undesirable sample images that generally lack depiction of tissue may facilitate improved processing and/or reduced wasted effort in processing, for example. In some embodiments, block 544 may direct the analyzer processor 400 to identify undesirable sample images based on alternative or additional analysis, such as, for example, identifying undesirable sample images as sample images that appear empty, do not represent any tissue, have only scanning artefacts (e.g. due to poor optical focus), have only folded (or otherwise damaged) tissue, generally have no useful tissue (e.g., only background and/or scanning artefacts and/or damaged tissue and/or other issues), or that are undesirable for analysis for another reason.

In various embodiments, identifying undesirable sample images may facilitate the property identifying neural network effectively skipping large non-tissue or other portions of the pathology image 102 for being undesirable. For example, in some embodiments, it may be possible to have the property identifying neural network effectively skip more than half or, in some examples, more than 90% of the sample image elements of the sample images, for example.

In some embodiments, block 544 may direct the analyzer processor 400 to cause the sample image elements of each sample image of the candidate set of sample images to be input into an undesirable image element detecting neural network. For example, in some embodiments, the undesirable image element detecting neural network may include a tissue detecting neural network. In some embodiments, using an undesirable image element detecting neural network and/or a tissue detecting neural network may facilitate improved identification of undesirable image elements.

In some embodiments, it may be possible and/or desirable to use a simpler neural network for the undesirable image element detecting neural network than what is used for the property identifying neural network (e.g., a tumor detecting neural network), and/or it may be possible and/or desirable to cause the undesirable image element detecting neural network to be able to process larger sample images at one time, and/or sample images corresponding to larger areas of the pathology image 102, compared to what is used for a property identifying neural network. Accordingly, in some embodiments, block 544 may direct the analyzer processor 400 to identify or determine sample images for analysis by the undesirable image element detecting neural network from the pathology image 102 for input into the undesirable image detecting neural network, the sample images for analysis by the undesirable image element detecting neural network being larger and fewer compared to the sample images described herein defined by the candidate sample image definition record 580 shown in FIG. 9 and stored in the location 442 of the storage memory 404.

In various embodiments, having the undesirable image element detecting neural network be substantially simpler and/or otherwise faster to process than the property identifying neural network may lead to notable performance benefits.

In some embodiments, block 544 may direct the analyzer processor 400 to determine a set of sample images for analysis by the undesirable image element detecting neural network. In some embodiments, block 544 may include generally similar code to that included in block 542 described above, but using a different sample image property record defining larger sample image height and width. In some embodiments, block 544 may direct the analyzer processor 400 to store a sample image definition record in the location 443 of the storage memory 404. In some embodiments, each sample image for analysis by the undesirable image element detecting neural network may correspond to an area of the pathology image 102 that is 20,000×20,000 pixels (i.e., 10× larger in each dimension than the sample images for analysis by a property identifying neural network or tumor detecting neural network). However, for processing (by the undesirable image element detecting neural network), each sample image may be downscaled from 20,000×20,000 to 2000×2000 pixels. In some embodiments, for example, the overlap could be 300 pixels in the coordinates of the pathology image 102, or 30 pixels in the coordinates of the downscaled sample images.

In some embodiments, the undesirable image element detecting neural network may have been previously defined and stored in the location 444 of the storage memory 404 shown in FIG. 5. In some embodiments, the undesirable image element detecting neural network may be configured to return a respective value for each sample image element of an input sample image, the value representing a determination whether the sample image element represents or lacks depiction of tissue.

In some embodiments, block 544 may direct the analyzer processor 400 to input sample images defined by the pathology image 102 and the sample image definition record stored in the location 443 of the storage memory 404 into a tissue detecting neural network acting as the undesirable image element detecting neural network and to generate contours of tissue sections in the pathology image 102 based on the output of the tissue detecting neural network. Next, block 544 may direct the analyzer processor 400 to compare the contours with the sample images defined by the candidate sample image definition record 580 shown in FIG. 9 and stored in the location 442 of the storage memory 404, to determine for each sample image defined by the candidate sample image definition record 580, whether the sample image contains any tissue. In various embodiments, if at block 544 it is determined that a sample image defined by the candidate sample image definition record 580 shown in FIG. 9 does not contain any tissue, then the analyzer processor 400 may identify the sample image as an undesirable sample image.

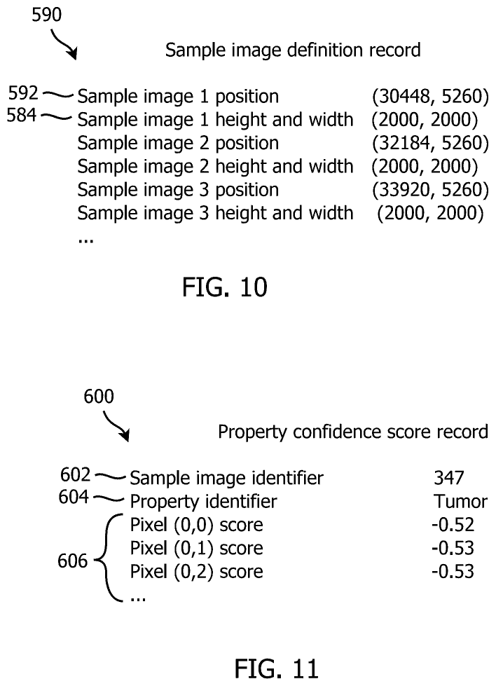

Referring to FIG. 7, in various embodiments, block 546 may direct the analyzer processor 400 to determine the plurality of sample images as a subset of the candidate set of sample images, the plurality of sample images not including the at least one undesirable sample image. In various embodiments, block 546 may direct the analyzer processor 400 to generate and store in the location 442 of the storage memory 404 a sample image definition record 590 as shown in FIG. 10. In various embodiments, block 546 may direct the analyzer processor 400 to include in the sample image definition record 590 only sample image fields or data from the candidate sample image definition record 580 shown in FIG. 9 when the sample image fields were associated with a sample image that was not identified as an undesirable image. Accordingly, in various embodiments, block 546 may direct the analyzer processor 400 to not include in the sample images identified and defined by the sample image definition record 590, any undesirable sample images as identified at block 544 of the flowchart 540 shown in FIG. 7.

After block 546 has been completed, the analyzer processor 400 may be directed to continue execution of the flowchart 500 shown in FIG. 6 at block 504.

Referring to FIG. 6, block 504 directs the analyzer processor 400 to cause one or more functions to be applied to the plurality of sample images to determine a plurality of confidence scores, each of the plurality of confidence scores associated with one of the plurality of sample image elements included in the sample images and representing a level of confidence that the associated sample image element represents the sample property.

In some embodiments, the one or more functions may include one or more property identifying neural network functions and block 504 may direct the image analyzer 12 to cause each of the sample images to be input into a property identifying neural network. In some embodiments, the one or more functions may include a traditional feature extractor function, e.g. HOG (Histogram of Oriented Gradients), accompanied by a classifier that can output something that can be interpreted as a confidence score, e.g. SVM (Support Vector Machine). In various embodiments, using a neural network function as the one or more functions may facilitate improved property identification and/or confidence determination.

In some embodiments, the one or more property identifying neural network functions may include a cancer or tumor detecting neural network for determining a tumor confidence score for each of a plurality of pixels included in the sample images, the tumor confidence score related to or meant to represent a level of confidence that a pixel represents a cancerous tumor. In such embodiments, the sample property may thus be “represents a tumor”. In various embodiments, other property identifying neural networks and/or property identifying neural network architectures may be used to determine further or alternative confidence scores acting as property specific confidence related scores for further or alternative properties including any or all of the properties set out herein.

In some embodiments, data defining the property identifying neural network may be stored in the location 445 of the storage memory 404. In some embodiments, the data defining the property identifying neural network may have been previously defined or determined during previous training, such as, for example, during training using expert evaluation of a plurality of sample images. In some embodiments, the property identifying neural network may have a U-Net architecture, for example.

In some embodiments, the property identifying neural network may have a limited field of view having a field of view width and a field of view height and the sample images may be overlapping by an overlap width and an overlap height between adjacent sample images, the overlap width and the overlap height chosen based on the field of view width and the field of view height. For example, in some embodiments, the overlap width may be greater than or equal to the field of view width minus one image element width and the overlap height may be greater than or equal to the field of view height minus one image element height. In various embodiments, the overlap width being greater than or equal to the field of view width minus one image element width and the overlap height being greater than or equal to the field of view height minus one image element height may facilitate use of full contextual consideration in the field of view for determining each pixel in the sample images. In some embodiments, the overlap width may be equal to the field of view width minus one image element width and the overlap height may be equal to the field of view height minus one image element height. In some embodiments, the overlap width being equal to the field of view width minus one image element width and the overlap height being equal to the field of view height minus one image element height may facilitate use of context with reduced sample image size to facilitate efficient processing of the sample images by the property identifying neural network.

Thus, in some embodiments, the property identifying neural network may have a limited field of view having a field of view width and a field of view height that is one pixel width and one pixel height greater than the overlap width and the overlap height respectively between adjacent sample images. Accordingly, in various embodiments, where each of the overlap width and height is 264 pixels, the property identifying neural network may have a limited field of view that is 265 pixels by 265 pixels in size for contextual consideration of each pixel.

In various embodiments, execution of block 504 may result in determining a tumor confidence score associated with each of the pixels of the sample images, the tumor confidence score associated with the property, “tumor” or “represents a tumor”. In various embodiments, block 504 may direct the analyzer processor 400 to store the tumor confidence scores in respective property confidence score records in the location 446 of the storage memory 404. In some embodiments, each of the property confidence score records may be generated based on one of the sample images and stored in association with the sample image from which it was generated.

In some embodiments, for example, block 504 may direct the analyzer processor 400 to generate and store a property confidence score record 600 as shown in FIG. 11 including a sample image identifier field 602 for storing an identifier for identifying the sample image from which the property confidence score record 600 was generated, a property identifier field 604 for storing an identifier for identifying the property associated with the scores included in the property confidence score record 600, and pixel score fields 606, each pixel score field associated with a pixel or pixel position included in the sample image from which the property confidence score record 600 was generated and storing a property confidence score related to or meant to represent a level of confidence that the pixel represents the property identified by the property identifier field 604. In some embodiments, for example, for the property confidence score record 600 shown in FIG. 11, the sample image identifier field 602 may store an identifier identifying the sample image 128 shown in FIG. 4 and the property identifier field 604 may store an identifier of “Tumor” for identifying the property as “Represents a tumor”.

Block 504 of the flowchart 500 shown in FIG. 6 may direct the analyzer processor 400 to generate and store a respective property confidence score record having the same format as the property confidence score record 600 shown in FIG. 11 in the location 446 of the storage memory 404 shown in FIG. 5, for each of the sample images defined by the sample image definition record 590 shown in FIG. 10. In various embodiments, the sample images defined by the sample image definition record 590 may include numerous sample images including the sample images 120-136 shown in FIG. 4.

Referring back to FIG. 6, block 506 directs the analyzer processor 400 to compare at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold. As described herein, in some embodiments, the candidate set of adjacent sample image elements may include sample image elements from more than one sample image. In various embodiments, the sample image elements coming from more than one sample image may facilitate more specific and/or more sensitive processing.

In some embodiments, a confidence score above the candidate confidence threshold may be necessary though not sufficient in determining that the associated sample image element represents a property. In some embodiments, if confidence scores for each sample image element of a candidate set of adjacent sample image elements are above the candidate confidence threshold and an additional test is met, it may be determined that the candidate set of adjacent sample image elements represent a property. However, if confidence scores for each sample image element of a candidate set of adjacent sample image elements are above the candidate confidence threshold and the additional test is not met, it may be that it is not determined that the candidate set of adjacent sample image elements represents the property.

In some embodiments, the additional test may require that a representative confidence score associated with the candidate set of adjacent sample image elements is greater than a confirmation confidence threshold. Thus, in some embodiments, if confidence scores for each sample image element of a candidate set of adjacent sample image elements are above the candidate confidence threshold and a representative confidence score associated with the candidate set of adjacent sample image elements is greater than a confirmation confidence threshold, it may be determined that the candidate set of adjacent sample image elements represent the property.

For example, in some embodiments, the representative confidence score may be a highest or greatest confidence score of the confidence scores for the candidate set of adjacent sample image elements and if the confidence scores for a candidate set of adjacent sample image elements are above the candidate confidence threshold and a highest or greatest confidence score of the confidence scores for the candidate set of adjacent sample image elements is greater than a confirmation confidence threshold, the confirmation confidence threshold being greater than the candidate confidence threshold, it may be determined that the candidate set of adjacent sample image elements represent the property.

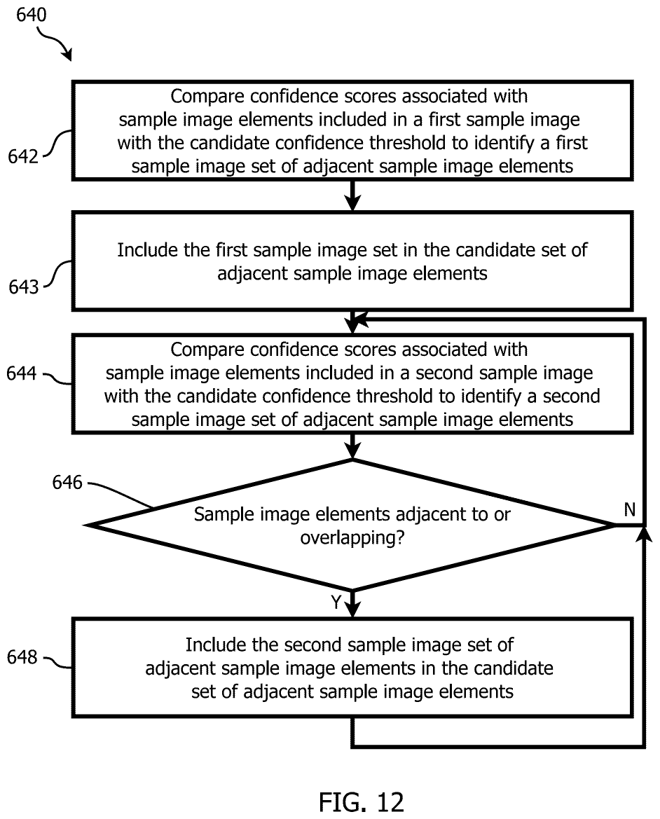

Referring to FIG. 12, there is shown a flowchart 640 depicting blocks of code that may be included in block 506 of the flowchart 500 shown in FIG. 6 in accordance with various embodiments. In various embodiments, blocks of code included in the flowchart 640 may direct the analyzer processor 400 to identify a candidate set of adjacent sample image elements, each sample image element of the candidate set of adjacent sample image elements associated with a confidence score greater than the candidate confidence threshold, wherein the candidate set of adjacent sample image elements includes sample image elements from more than one of the sample images.

The flowchart 640 begins with block 642, which directs the analyzer processor 400 to compare confidence scores associated with sample image elements included in a first sample image with the candidate confidence threshold to identify a first sample image set of adjacent sample image elements, each sample image element of the first sample image set of adjacent sample image elements associated with a confidence score greater than the candidate confidence threshold.

In some embodiments, the first sample image may be chosen from the sample images defined by the pathology image data stored in the location 440 of the storage memory 404 shown in FIG. 5 and the sample image definition record 590 shown in FIG. 10 and stored in the location 442 of the storage memory 404. In various embodiments, the flowchart 640 may be repeatedly executed to consider each of the sample images defined by the sample image definition record 590 shown in FIG. 10 in turn as the first sample image.

In some embodiments, during an execution of block 642, the first sample image may be the sample image 128 shown in FIG. 4 and block 642 may direct the analyzer processor 400 to read the property confidence score record 600 shown in FIG. 11 from the location 446 of the storage memory 404 and compare each confidence score with the candidate confidence threshold. In some embodiments, the candidate confidence threshold may have previously been provided and may be stored in the location 448 of the storage memory 404. For example, in some embodiments, the candidate confidence threshold may be set to a value of 0.0. In some embodiments, the candidate confidence threshold may be another value, such as, for example, 0.5. In some embodiments, the confidence scores may be limited to a range from −1.0 to +1.0, for example. In some embodiments, the confidence scores may be distributed with no strict upper or higher limit (e.g., a normal distribution), such that, the further from zero the value, the more confidence there is that the corresponding pixel either represents (>0) or does not represent (<0) a property.

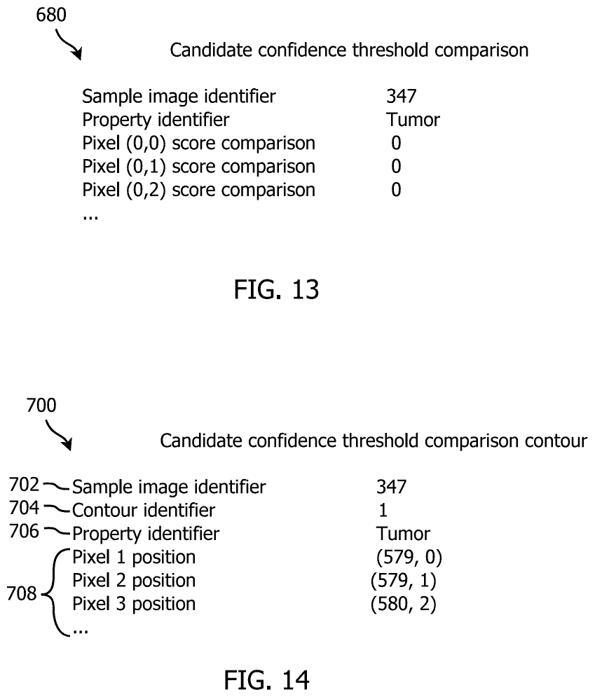

Block 642 may direct the analyzer processor 400 to generate a binary image representation of the result of the comparison of each confidence score of the property confidence score record 600 shown in FIG. 11, wherein each pixel position associated with a confidence score greater than or equal to the candidate confidence threshold is set to 1 and each pixel position associated with a confidence score less than the candidate confidence threshold is set to 0. In various embodiments, block 642 may direct the analyzer processor 400 to store a candidate confidence threshold comparison record 680 as shown in FIG. 13 representing the binary image in the location 450 of the storage memory 404. In various embodiments, the candidate confidence threshold comparison record 680 may represent one or more sets of adjacent sample image elements, with each of the sets of adjacent sample image elements representing a continuous region of adjacent pixels in the sample image.

In some embodiments, block 642 may direct the analyzer processor 400 to identify and group together sample image elements associated with confidence scores greater than the candidate confidence threshold. Accordingly, in various embodiments, block 642 may direct the analyzer processor 400 to identify sets of adjacent sample image elements from the candidate confidence threshold comparison record 680 stored in the location 450 of the storage memory 404. In some embodiments, for example, block 642 may direct the analyzer processor 400 to extract contours from the binary image represented by the candidate confidence threshold comparison record. For example, in some embodiments, block 642 may direct the analyzer processor 400 to use OpenCV's findContours function to extract the contours. Block 642 may direct the analyzer processor 400 to store the extracted contours in the location 450 of the storage memory 404. Referring to FIG. 14, there is shown a candidate confidence threshold comparison contour record 700 for storing a representation of an extracted contour, which may be stored in the location 450 of the storage memory 404, in accordance with various embodiments. In various embodiments, the candidate confidence threshold comparison contour record 700 may act as an identifier of a first sample image set of adjacent sample image elements, wherein the first sample image set of adjacent sample image elements are contained within a contour defined by the candidate confidence threshold comparison contour record 700 when applied to the sample image 128. In some embodiments, block 642 may direct the analyzer processor 400 to generate and store in the location 450 of the storage memory 404 an additional candidate confidence threshold comparison contour record for each set of adjacent sample image elements identified from the binary image represented by the candidate confidence threshold comparison record 680 shown in FIG. 13.

Referring to FIG. 14, the candidate confidence threshold comparison contour record 700 includes a sample image identifier field 702 for storing an identifier of the sample image from which the contour was generated, a contour identifier field 704 for storing an identifier of the contour, which may be a unique contour identifier for the sample image and property identified, and a property identifier field 706 for storing an identifier of the property to which the candidate confidence threshold comparison contour record 700 relates. In various embodiments, the sample image identifier field 702, the contour identifier field 704, and the property identifier field 706 may together uniquely identify the candidate confidence threshold comparison contour record 700 from other candidate confidence threshold comparison contour records stored in the location 450 of the storage memory 404 shown in FIG. 5.

Referring still to FIG. 14, the candidate confidence threshold comparison contour record 700 includes pixel position fields 708 for storing pixel positions defining a shape of the contour defined by the candidate confidence threshold comparison contour record 700 applied to the sample image identified in the sample image identifier field 702.

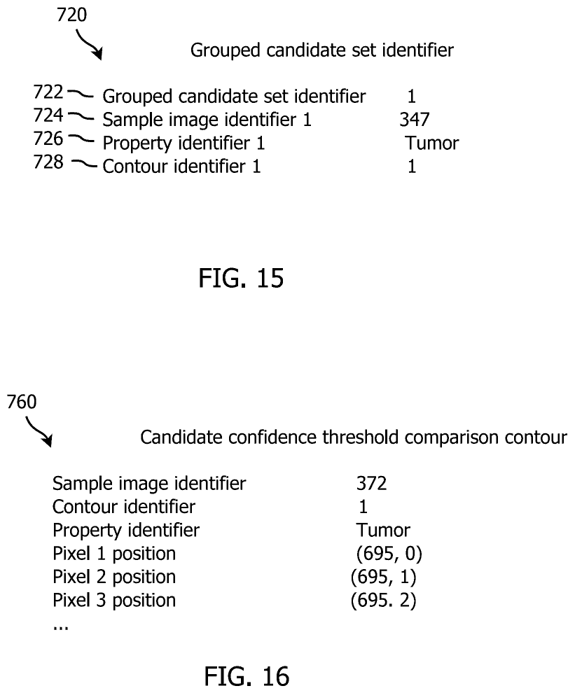

Referring back to FIG. 12, block 643 directs the analyzer processor 400 to include the first sample image set of adjacent sample image elements in the candidate set of adjacent sample image elements. For example, in some embodiments, block 643 may direct the analyzer processor 400 to generate a grouped candidate set identifier record 720 as shown in FIG. 15, the grouped candidate set identifier record 720 identifying one or more contours, each of the contours identifying sets of sample image elements included in a candidate set of adjacent sample image elements.

Referring to FIG. 15, the grouped candidate set identifier record 720 includes a grouped candidate set identifier field 722 for storing an identifier of the candidate set of adjacent sample image elements and contour identification fields for identifying one or more contours identifying sets of sample image elements included in the candidate set of adjacent sample image elements. In some embodiments, the grouped candidate set identifier record 720 may include a first sample image identifier field 724, a first contour property identifier field 726, and a first contour identifier field 728 for storing, respectively, an identifier of a sample image, a property identifier, and a contour identifier, to uniquely identify a contour defined by a candidate confidence threshold comparison contour record. In various embodiments, the first sample image identifier field 724, the first contour property identifier field 726, and the first contour identifier field 728 of the grouped candidate set identifier record 720 shown in FIG. 15 may store values uniquely identifying the contour defined by the candidate confidence threshold comparison contour record 700 shown in FIG. 14.

In various embodiments, block 643 may direct the analyzer processor 400 to store the grouped candidate set identifier record 720 in the location 452 of the storage memory 404 shown in FIG. 5. In some embodiments block 643 may direct the analyzer processor 400 to generate and store a respective grouped candidate set identifier record for each of the candidate confidence threshold comparison contour records stored in the location 450 of the storage memory 404.

Referring back to FIG. 12, the flowchart 640 may include code for directing the analyzer processor 400 to perform generally similar steps to those set out having regard to block 642, but with a different sample image that is adjacent to the first sample image. Accordingly, in various embodiments, the flowchart 640 may include block 644, which directs the analyzer processor 400 to compare confidence scores associated with sample image elements included in a second sample image with the candidate confidence threshold to identify a second sample image set of adjacent sample image elements. In various embodiments, block 644 may direct the analyzer processor 400 to perform generally similar steps to those set out having regard to block 642, but using the sample image 134 shown in FIG. 4. Accordingly, after block 644 has been executed, a candidate confidence threshold comparison contour record 760 as shown in FIG. 16 may be stored in the location 450 of the storage memory 404.

Referring to FIG. 12, block 646 then directs the analyzer processor 400 to determine whether at least one sample image element of the first sample image set is adjacent to or overlapping (e.g., intersecting) with at least one sample image element of the second sample image set. In various embodiments, if the sample image sets are adjacent or overlapping, then they may be considered as a single set of sample image elements that continues across more than one sample image.

In various embodiments, block 646 may direct the analyzer processor 400 to compare the contours represented by the candidate confidence threshold comparison contour record 700 shown in FIG. 14 and the candidate confidence threshold comparison contour record 760 shown in FIG. 16 to determine whether the contours overlap. In various embodiments, block 646 may direct the analyzer processor 400 to read the image position or offset for the respective sample images 128 and 134 shown in FIG. 4 from the location 442 of the storage memory 404 shown in FIG. 5 to determine global pixel positions for the pixel positions stored in the candidate confidence threshold comparison contour records 700 and 760 shown in FIGS. 14 and 16 when determining whether the contours overlap.

In various embodiments, execution of block 646 may direct the analyzer processor 400 to cause an is-intersecting function and/or an is-touching function, for example, to be performed to determine whether the contours represented by the candidate confidence threshold comparison contour records 700 and 760 shown in FIGS. 14 and 16 are adjacent or overlapping.

In various embodiments, if at block 646 the analyzer processor 400 determines that at least one sample image element of the first sample image set is adjacent to or overlapping with at least one sample image element of the second sample image set, then the analyzer processor 400 is directed to proceed to block 648. For example, in some embodiments, block 646 may direct the analyzer processor 400 to proceed to block 648 after the intersect function applied to the contours represented by the candidate confidence threshold comparison contour records 700 and 760 shown in FIGS. 14 and 16 returns a result indicating that the contours overlap.

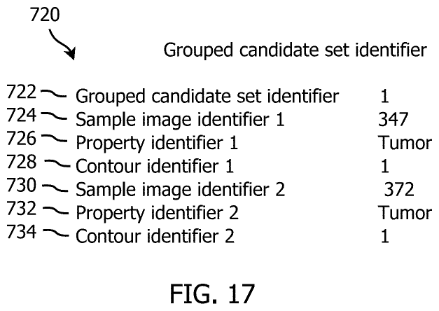

Block 648 directs the analyzer processor 400 to include the second sample image set of adjacent sample image elements in the candidate set of adjacent sample image elements. Accordingly, block 648 directs the analyzer processor 400 to combine the first and second sample image sets of adjacent sample image elements into a single set of adjacent sample image elements. In various embodiments, block 648 may direct the analyzer processor 400 to update the grouped candidate set identifier record 720 shown in FIG. 15 and stored in the location 452 of the storage memory 404 to include an identification of the candidate confidence threshold comparison contour record 760 shown in FIG. 16. For example, in some embodiments, block 648 may direct the analyzer processor 400 to add a second sample image identifier field 730, a second contour property identifier field 732, and a second contour identifier field 734 as shown in FIG. 17 for storing identifiers uniquely identifying the contour defined by the candidate confidence threshold comparison contour record 760 shown in FIG. 16.

In some embodiments, blocks 646 and 648 may be repeated considering each candidate confidence threshold comparison contour record that is stored in the location 450 of the storage memory 404 and associated with the sample image 134 and the same property identifier of “Tumor” as the candidate confidence threshold comparison contour record 700 shown in FIG. 14, compared to the candidate confidence threshold comparison contour record 700 shown in FIG. 14.

In some embodiments, blocks 646 and 648 may be repeated considering each candidate confidence threshold comparison contour record identified by a grouped candidate set identifier record and associated with the sample image 128, compared to each candidate confidence contour record associated with the sample image 134 that is associated with the same property and stored in the location 450 of the storage memory 404.

In some embodiments, if at block 646, the analyzer processor 400 does not determine that at least one sample image element of the first sample image set is adjacent to or overlapping with at least one sample image element of the second sample image set or after execution of block 648, the analyzer processor 400 may be directed to return to block 644 and to consider a further sample image as the second sample image. In various embodiments, blocks 644, 646 and 648 may be repeated for all sample images that are adjacent to the sample image 128 considered as the first sample image at block 642.

In various embodiments, blocks 642 and/or 644 may be executed for different sample images in parallel, which may speed up processing and facilitate getting the results faster. In some embodiments, candidate confidence threshold comparison contour records may be generated in parallel for all of the sample images before performing comparisons and/or intersection determinations to determine whether the contours are adjacent or overlapping and then the analyzer processor 400 may be directed to group adjacent or overlapping contours via respective grouped candidate set identifier records.

In various embodiments, after all sample images adjacent to the sample image 128 are considered there may be stored in the location 452 of the storage memory 404 shown in FIG. 5, one or more grouped candidate set identifier records, each having generally similar format to the grouped candidate set identifier record 720 shown in FIG. 17. In various embodiments, a flowchart generally similar to the flowchart 640 may be executed to consider all sample images and adjacent sample images defined by the sample image definition record 590 shown in FIG. 10 and stored in the location 442 of the storage memory 404. In various embodiments, a block of code included in the flowchart 640 shown in FIG. 12 or included in block 506 of the flowchart 500 shown in FIG. 6 may direct the analyzer processor 400 to cause grouped candidate set identifier records that include a common contour to be combined into a single grouped candidate set identifier record.

Referring back to FIG. 6, block 508 directs the analyzer processor 400 to determine whether at least one representative confidence score associated with the candidate set of adjacent sample image elements is greater than a confirmation confidence threshold. In some embodiments, block 508 may direct the analyzer processor 400 to determine the at least one representative confidence score based on the confidence scores associated with the candidate set of adjacent sample image elements. In various embodiments, this may facilitate use of the confirmation confidence threshold as a second dimension for checking the confidence scores associated with the candidate set of adjacent sample image elements, which may facilitate an increase in specificity with reduced sacrifice of sensitivity of the analysis, may facilitate an increase in sensitivity with reduced sacrifice of specificity, and/or may improve both specificity and sensitivity. In some embodiments, block 508 may direct the analyzer processor 400 to identify the at least one representative confidence score from the confidence scores associated with the candidate set of adjacent sample image elements. In some embodiments, the confirmation confidence threshold may be greater than the candidate confidence threshold. In various embodiments, use of a confirmation confidence threshold that is greater than the candidate confidence threshold in the context of a representative confidence score may facilitate an increase in specificity and/or sensitivity of the analysis without significant reduction in either specificity or sensitivity.

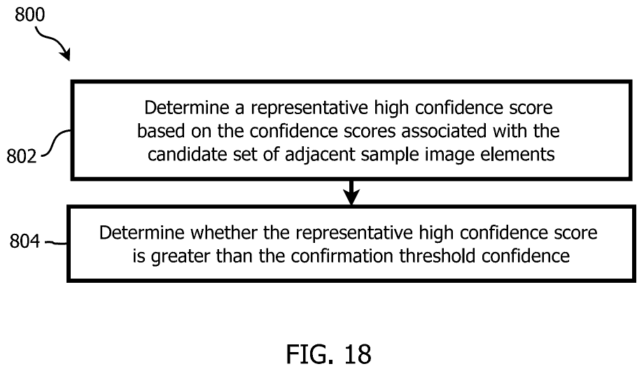

Referring to FIG. 18, there is shown a flowchart 800 depicting blocks of code that may be included in block 508 of the flowchart 500 shown in FIG. 6 in accordance with various embodiments.

In some embodiments, the at least one representative score may include a representative high confidence score and the flowchart 800 begins with block 802, which directs the analyzer processor 400 to determine a representative high confidence score based on the confidence scores associated with the candidate set of adjacent sample image elements. In various embodiments, the representative high confidence score may be identified in various ways. In some embodiments, the representative high confidence score may be identified based on comparison or analysis of confidence scores from more than one sample image and may facilitate an increase in specificity and/or sensitivity of analysis of a large pathology image without significant reduction in either specificity or sensitivity.

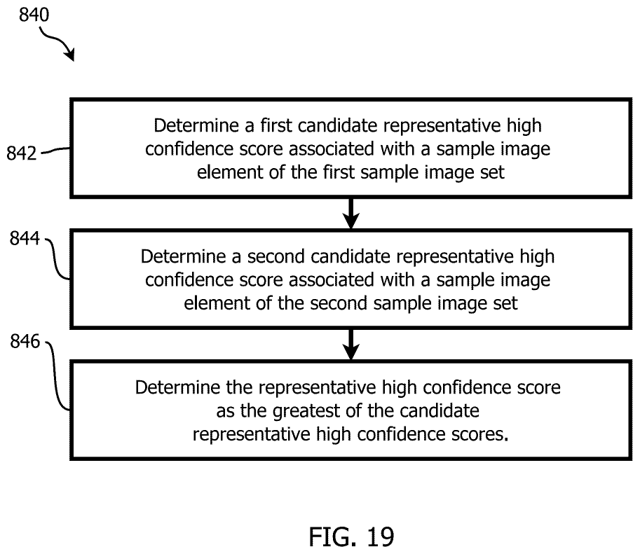

Referring to FIG. 19, there is shown a flowchart 840 depicting blocks of code that may be included in block 802 of the flowchart 800 shown in FIG. 18 in accordance with various embodiments. Referring to FIG. 19, the flowchart 840 begins with block 842, which directs the analyzer processor 400 to determine a first candidate representative high confidence score associated with a sample image element of the first sample image set.

In various embodiments, block 842 may direct the analyzer processor 400 to read the grouped candidate set identifier records stored in the location 452 of the storage memory 404 shown in FIG. 5. For example, in some embodiments, block 842 may direct the analyzer processor 400 to read the grouped candidate set identifier record 720 stored in the location 452 of the storage memory 404 shown in FIG. 5 and to use the first sample image identifier field 724, the first contour property identifier field 726, and the first contour identifier field 728 to identify the candidate confidence threshold comparison contour record 700 shown in FIG. 14 and stored in the location 450 of the storage memory 404. Block 842 may direct the analyzer processor 400 to use the pixel position values stored in the candidate confidence threshold comparison contour record 700 to identify pixels or pixel positions included in the sample image 128 as sample image elements of the first sample image set. In various embodiments, block 842 may direct the analyzer processor 400 to identify confidence scores associated with each pixel using the property confidence score record 600 shown in FIG. 11 and stored in the location 446 of the storage memory 404.

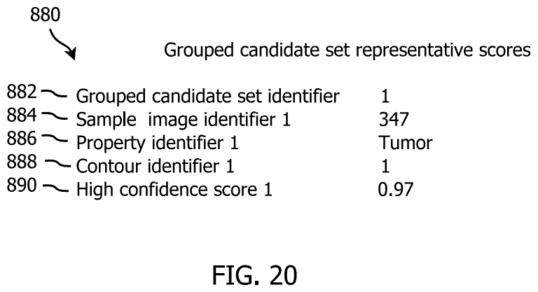

Block 842 then directs the analyzer processor 400 to compare the confidence scores associated with each identified pixel to determine the first candidate representative high confidence score. For example, in some embodiments, block 842 may direct the analyzer processor 400 to determine the first candidate representative high confidence score to be the greatest value confidence score associated with one of the identified pixels. In various embodiments, block 842 may direct the analyzer processor 400 to generate and store in the location 454 of the storage memory 404, a grouped candidate set representative scores record 880 as shown in FIG. 20 including the first candidate representative high confidence score.

In various embodiments, the grouped candidate set representative scores record 880 may include a grouped candidate set identifier field 882, a first sample image identifier field 884, a first contour property identifier field 886, and a first contour identifier field 888. The grouped candidate set representative scores record 880 may also include a first high confidence score field 890, associated with the fields 884, 886, and 888, for storing a first candidate representative high confidence score. In various embodiments, block 842 may direct the analyzer processor 400 to store the determined first candidate representative high confidence score in the first high confidence score field 890 of the grouped candidate set representative scores record 880 shown in FIG. 20.

Referring back to FIG. 19, block 844 directs the analyzer processor 400 to determine a second candidate representative high confidence score associated with a sample image element of the second sample image set. In various embodiments, block 844 may include code generally similar to that of block 842 but directing the analyzer processor 400 to consider the candidate confidence threshold comparison contour record 760 shown in FIG. 16 and the sample image 134 identified therein to determine the second candidate representative high confidence score. For example, in some embodiments, block 844 may direct the analyzer processor 400 to determine the second candidate representative high confidence score to be the greatest value confidence score associated with the identified pixels of the sample image 134.

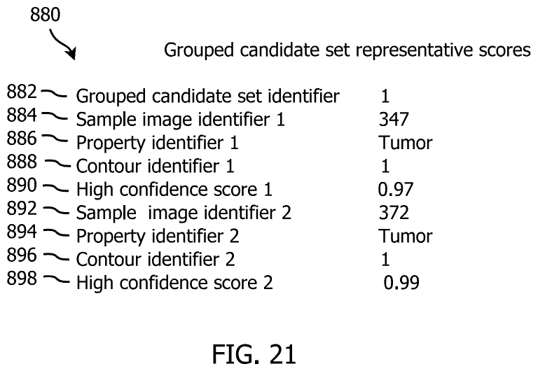

In various embodiments, block 844 may direct the analyzer processor 400 to update the grouped candidate set representative scores record 880 stored in the location 454 of the storage memory 404 shown in FIG. 5 to include a second sample image identifier field 892, a second contour property identifier field 894, a second contour identifier field 896, and an associated second high confidence score field 898 as shown in FIG. 21.

In various embodiments, where a grouped candidate set identifier record similar to the grouped candidate set identifier record 720 shown in FIG. 17 may include further contour identifier fields (for example, from alternative sample images), the flowchart 840 shown in FIG. 19 may include further blocks of code generally similar to the blocks 842 and 844 to consider each of the contours identified in the grouped candidate set identifier record and to direct the analyzer processor 400 to update the grouped candidate set representative scores record 880 shown in FIG. 21 stored in the location 454 of the storage memory 404 shown in FIG. 5 to include further sample image identifier fields, contour property identifier fields, contour identifier fields, and associated high confidence score fields.

In various embodiments, considering candidate representative high confidence scores from more than one sample image may facilitate efficient, specific, and/or sensitive analysis applied to large pathological images and/or image data.

Referring to FIG. 19, in various embodiments, block 846 may direct the analyzer processor 400 to determine the representative high confidence score as the greatest of the candidate representative high confidence scores. For example, in some embodiments, block 846 may direct the analyzer processor 400 to determine the representative high confidence score as the greatest of the first and second candidate representative high confidence scores.

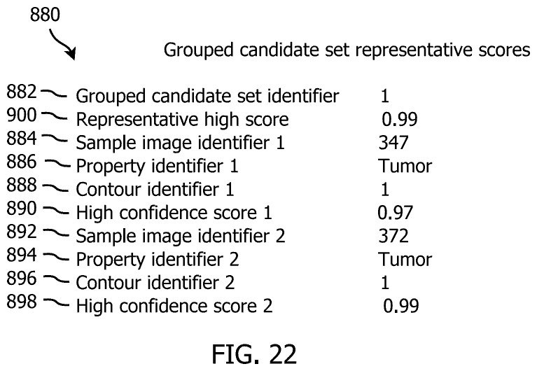

In some embodiments, block 846 may direct the analyzer processor 400 to read the high confidence score fields of the grouped candidate set representative scores record 880 and to determine the greatest of the high confidence scores stored in the high confidence score fields. In various embodiments, block 846 may direct the analyzer processor 400 to update the grouped candidate set representative scores record 880 stored in the location 454 of the storage memory 404 shown in FIG. 5 to include a representative high confidence score field 900 for storing the determined greatest of the high confidence scores as shown in FIG. 22. In various embodiments, block 846 may direct the analyzer processor 400 to store the determined greatest of the high confidence scores in the representative high confidence score field 900 of the grouped candidate set representative scores record 880 stored in the location 454 of the storage memory 404 shown in FIG. 5.

In various embodiments, determining the representative high confidence score from different candidate high confidence scores may facilitate parallel processing, which may facilitate more efficient analysis.

In various embodiments, after execution of block 846 of the flowchart 840 shown in FIG. 19, the analyzer processor 400 may be directed to proceed to block 804 of the flowchart 800 shown in FIG. 18. Referring to FIG. 18, block 804 directs the analyzer processor 400 to determine whether the representative high confidence score is greater than a confirmation confidence threshold. In some embodiments, the confirmation confidence threshold may be greater than the candidate confidence threshold. In various embodiments, the confirmation confidence threshold may have been previously provided and stored in the location 456 of the storage memory 404 shown in FIG. 5. For example, in some embodiments, the confirmation confidence threshold may be stored as a value of 0.8. In various embodiments, the candidate and/or confirmation confidence thresholds may have been previously determined by expert evaluation. For example, in some embodiments, an experienced pathologist may look at various results, and based on experience and/or results/outcomes, choose the candidate and/or confirmation confidence thresholds such that there is a desired or suitable trade-off between specificity and sensitivity.

In some embodiments, block 804 may direct the analyzer processor 400 read the representative high confidence score field 900 of the grouped candidate set representative scores record 880 shown in FIG. 22 and stored in the location 454 of the storage memory 404 shown in FIG. 5 and compare the representative high confidence score stored therein with the confirmation confidence threshold stored in the location 456 of the storage memory 404 to determine whether the representative high confidence score is greater than the confirmation confidence threshold.

In various embodiments, if at block 804 of the flowchart 800 shown in FIG. 18, the analyzer processor 400 determines that the representative high confidence score is greater than the confirmation confidence threshold, then this may act as a determination in block 508 of the flowchart 500 shown in FIG. 6 that the at least one representative confidence score associated with the candidate set of adjacent sample image elements is greater than the confirmation confidence threshold and the analyzer processor 400 may be directed to proceed to block 510 of the flowchart 500.

In various embodiments, having the flowchart 800 direct the analyzer processor 400 to determine a representative high confidence score based on the confidence scores associated with the candidate set of adjacent sample image elements and determine whether the representative high confidence score is greater than the confirmation confidence threshold may facilitate a second threshold being applied based on different criteria compared to the first threshold, and/or may facilitate an increase in specificity and/or sensitivity of pathological analysis without significant reduction in either specificity or sensitivity.

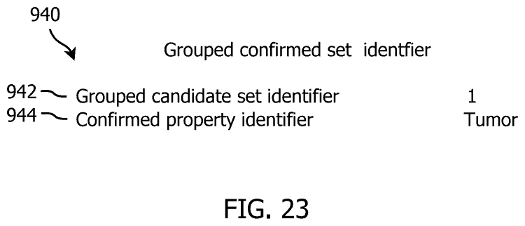

Referring to FIG. 6, block 510 directs the analyzer processor 400 to associate the candidate set of adjacent sample image elements with a sample property identifier for identifying the candidate set of adjacent sample image elements as representing the sample property. In some embodiments, block 510 may direct the analyzer processor 400 to generate a grouped confirmed set identifier record 940 as shown in FIG. 23, the grouped confirmed set identifier record 940 identifying and associating a candidate set of adjacent sample image elements with a sample property identifier for identifying the candidate set of adjacent sample image elements as representing the sample property. Referring to FIG. 23, the grouped confirmed set identifier record 940 may include a grouped candidate set identifier field 942 for storing an identifier identifying a grouped candidate set identifier record and a confirmed property identifier field 944 for storing a property identifier. In various embodiments, block 510 may direct the analyzer processor 400 to set the grouped candidate set identifier field 942 to correspond to the identifier stored in the grouped candidate set identifier field 882 of the grouped candidate set representative scores record 880 considered at block 508, upon which the decision to proceed to block 510 was based. In various embodiments, block 510 may direct the analyzer processor 400 to store the grouped confirmed set identifier record 940 in the location 458 of the storage memory 404 shown in FIG. 5.

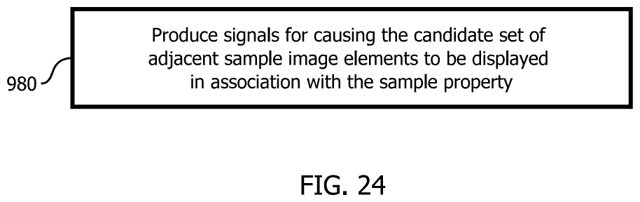

In some embodiments, block 510 may include the block of codes 980 shown in FIG. 24, which directs the analyzer processor 400 to produce signals for causing the candidate set of adjacent sample image elements to be displayed in association with the sample property. In some embodiments, block 980 may be executed upon receiving a request initiated by a pathologist viewing the display 16 shown in FIG. 1. For example, in some embodiments, the request may have been received by the image analyzer 12 via an input device communicating with the I/O interface 412 of the image analyzer 12 shown in FIG. 5.

In some embodiments, block 510 may direct the analyzer processor 400 to transmit or send signals to the display 16 representing at least a portion of the pathology image 102 shown in FIG. 2 and the candidate set of adjacent sample image elements associated with the grouped confirmed set identifier record 940 via the interface 422 of the I/O interface 412 shown in FIG. 5.

For example, in some embodiments, block 510 may direct the analyzer processor 400 to identify the sample image elements by reading all grouped confirmed set identifier records stored in the location 458 of the storage memory 404, including the grouped confirmed set identifier record 940 shown in FIG. 23, from the location 458 of the storage memory 404 shown in FIG. 5; using the grouped candidate set identifier field from each grouped confirmed set identifier record to identify and read a grouped candidate set identifier record, such as the grouped candidate set identifier record 720 shown in FIG. 17, from the location 452 of the storage memory 404; and using the sets of associated image identifier fields, property identifier fields, and contour identifier fields from each grouped candidate set identifier record to identify and read respective candidate confidence threshold comparison contours, such as the candidate confidence threshold comparison contour records 700 and 760 shown in FIGS. 14 and 16. In various embodiments, block 510 may direct the analyzer processor 400 to send pathology image data and a representation of the identified candidate confidence threshold comparison contour records to the display 16 shown in FIG. 1 via the interface 422 of the I/O interface 412 shown in FIG. 5.

In some embodiments, block 510 may direct the analyzer processor 400 to join the contours represented by the candidate confidence threshold comparison contour records 700 and 760 shown in FIGS. 14 and 16 before sending a representation of the joined contours. For example, in some embodiments, block 510 may direct the analyzer processor 400 to perform a union operation on the candidate confidence threshold comparison contour records 700 and 760 and to send a representation of the result of the union operation to the display 16 shown in FIG. 1 via the interface 422 of the I/O interface 412 shown in FIG. 5.

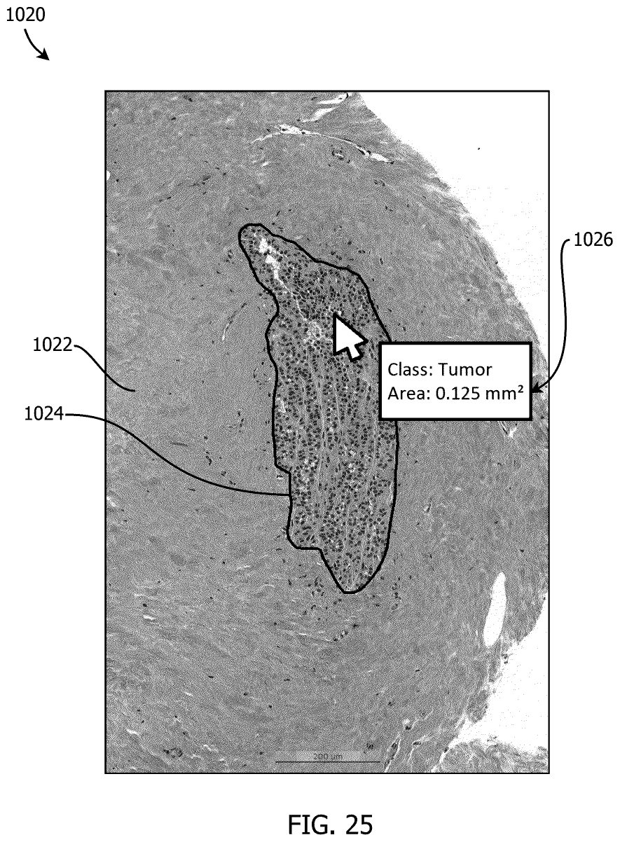

In various embodiments, in response to receiving the signals representing at least a portion of the pathology image data and the candidate set of adjacent sample image elements, the display 16 may provide a visual representation of pathology image data and the identified candidate confidence threshold comparison contour records as shown at 1020 in FIG. 25. Referring to FIG. 25, the visual representation 1020 includes a representation 1022 of a portion of the pathology image data and a representation 1024 of the union of the candidate confidence threshold comparison contour records 700 and 760 shown in FIGS. 14 and 16 as identified using the grouped confirmed set identifier record 940 shown in FIG. 23.

In various embodiments, producing signals for causing the candidate set of adjacent sample image elements to be displayed in association with the sample property may facilitate analysis, diagnoses, and/or treatment decisions by a user or pathologist viewing the display 16.

In some embodiments, block 510 may direct the analyzer processor 400 to derive or determine metrics related to the candidate set of adjacent sample image elements. For example, in some embodiments, block 510 may direct the analyzer processor 400 to determine a total area of the candidate set of adjacent sample image elements. In some embodiments, block 510 may direct the analyzer processor 400 to determine a total area of the union of the candidate confidence threshold comparison contour records 700 and 760, for example.

In various embodiments, block 510 may direct the analyzer processor 400 to produce signals for causing the metrics to be displayed to a user (e.g., a pathologist). In some embodiments, block 510 may direct the analyzer processor 400 to cause signals representing the determined area to be sent to the display 16 for causing the display 16 to display the determined area as shown at 1026 in FIG. 25, for example. In some embodiments, the determined area may be shown as a tooltip when a cursor or pointer is hovered inside the tumor contour, for example.

In some embodiments, block 510 may direct the analyzer processor 400 to generate a report including the determined metrics and to cause signals representing the report to be sent to the display 16 for causing the display 16 to display the report.

In some embodiments, the report and/or the metrics displayed may affect treatment decisions (e.g., surgery, chemotherapy, radiation, and/or another treatment decision).

In some embodiments, additional or alternative metrics may be determined, such as, for example, grade of tumor, and block 510 may direct the analyzer processor 400 to produce signals for causing the additional or alternative metrics to be displayed to a user (e.g., a pathologist). In some embodiments, block 510 may direct the analyzer processor 400 to input the candidate set of adjacent sample image elements and/or additional contextual image data into a tumor grade determining neural network trained to output a detection and/or classification result representing a tumor grade for the candidate set of adjacent sample image elements. In various embodiments, the tumor grade determining neural network may be trained, for example by first manually annotating each tumor (or part of tumor) in the training data accordingly (e.g., as a “grade 3” or “grade 4” or “grade 5” tumor), and then training the tumor grade determining neural network based on the manually annotated training data.

In some embodiments, block 510 may direct the analyzer processor 400 to produce signals for causing an automated treatment recommendation and/or decision to be performed. In various embodiments, a human expert may review and manually override the automated treatment decision.

In various embodiments, block 510 may direct the analyzer processor 400 to use the determined metrics to automatically prioritize review of patient cases and/or treatment for patients. In some embodiments, a relatively higher-grade tumor area may lead to a patient case receiving a relatively higher priority, for example. In various embodiments, a human expert may review and manually override the automatic prioritization of patient cases. In some embodiments, block 510 may direct the analyzer processor 400 to produce signals for causing a worklist to be generated and/or displayed to a user, wherein the worklist includes patient cases listed or ordered in a prioritized order based on one or more of the determined metrics (e.g., ordered such that patient cases having higher grade tumors are listed or ranked first).

In various embodiments, the worklist may be generated based on availability and/or expertise of candidate sample reviewing experts, such as pathologists. For example, in some embodiments, block 510 may direct the analyzer processor 400 to produce signals for generating a worklist wherein relatively difficult patient cases are to be directed to relatively experienced sample reviewing experts, and relatively easy patient cases are to be directed to relatively inexperienced sample reviewing experts. For example, in some embodiments, borderline cases where the accuracy of the image analysis results may critically affect the patient being eligible/non-eligible for a specific treatment may be considered as relatively difficult patient cases and may be directed to relatively more experienced sample reviewing experts, and some clearly positive or negative cases may be directed to any expert.

In various embodiments, block 510 may direct the analyzer processor 400 to produce signals for causing action to be taken. For example, in some embodiments, block 510 may direct the analyzer processor 400 to produce signals for causing at least one new sample staining to be ordered from a laboratory. In some embodiments, if a tumor has been detected, some additional biomarker stainings may be ordered automatically from the sample handling laboratory. In some embodiments, block 510 may direct the analyzer processor 400 to transmit a message to the LIS 18 via the interface 424 of the I/O interface 412, the message including a patient ID, sample ID, stain type, an indication that the patient has a cancerous tumor, and/or an area of the tumor. In various embodiments, the LIS 18 may be configured to receive the message and cause at least one additional biomarker staining to be ordered based on the message.

### VARIOUS EMBODIMENTS

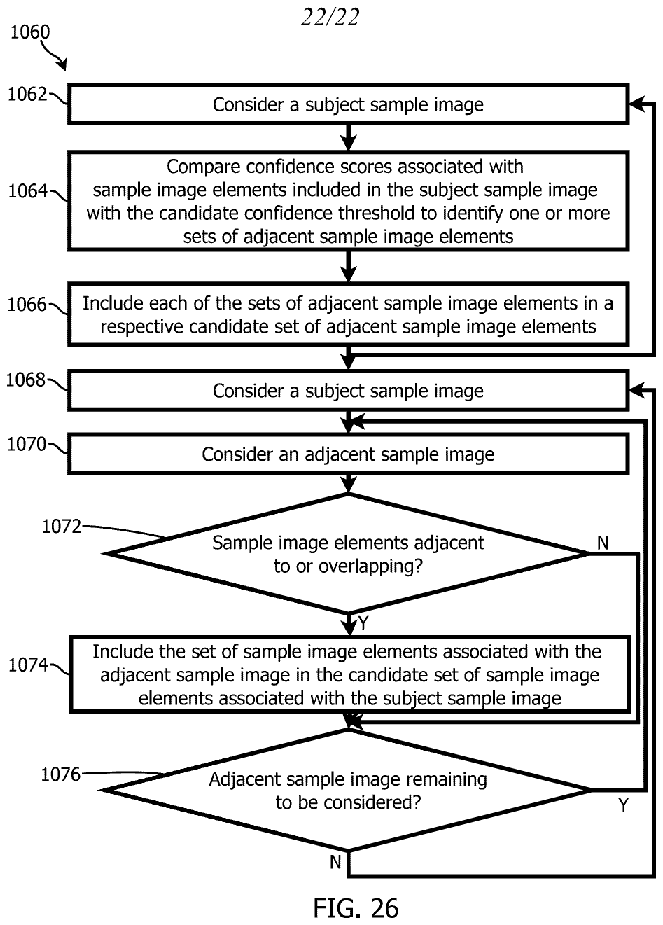

In various embodiments, the organization and/or order of operation for executing the blocks of code of the flowchart 640 shown in FIG. 12 may be varied and/or incorporated into a larger process and/or blocks of code. For example, in some embodiments, the block of codes 506 of the flowchart 500 shown in FIG. 6 may include blocks of code of a flowchart 1060 as shown in FIG. 26. In some embodiments, the flowchart 1060 may incorporate functionality and/or code described above having regard to the flowchart 640 shown in FIG. 12.

Referring to FIG. 26, the flowchart 1060 begins with blocks 1062, 1064, and 1066, which may be repeated to cause the analyzer processor 400 to consider each of the sample images to identify respective sample image sets of adjacent sample image elements, each sample image element of the sample image sets associated with a confidence score greater than the candidate confidence threshold, and to include each of the sample image sets in a respective candidate set of adjacent sample image elements.

In various embodiments, block 1062 may direct the analyzer processor 400 to consider a subject sample image. In some embodiments, the first sample image considered at block 1062 may be a sample image in the top left of the pathology image 102 shown in FIG. 3, for example. However, in some embodiments, the first sample image considered may be taken from another location in the pathology image 102, such as, from the middle of the image.

In various embodiments, block 1064 then directs the analyzer processor 400 to compare confidence scores associated with sample image elements included in the subject sample image with the candidate confidence threshold to identify one or more sets of adjacent sample image elements, each sample image element of the one or more sets of adjacent sample image elements associated with a confidence score greater than the candidate confidence threshold.

In various embodiments, block 1064 may include code generally similar to that included block 642 of the flowchart 640 shown in FIG. 12.

Referring to FIG. 26, in various embodiments, block 1066 may direct the analyzer processor 400 to include each of the identified sets of adjacent sample image elements in a respective candidate set of adjacent sample image elements.

In various embodiments, block 1066 may include code generally similar to that of block 643 of the flowchart 640 shown in FIG. 12, such that one or more candidate confidence threshold comparison contour records generally similar to the candidate confidence threshold comparison contour record 700 shown in FIG. 14 are stored in the location 450 of the storage memory 404 shown in FIG. 5 and such that one or more grouped candidate set identifier record generally similar to the grouped candidate set identifier record 720 as shown in FIG. 15 are stored in the location 452 of the storage memory 404.

In various embodiments, after block 1066 is executed, the analyzer processor 400 may be directed to return to block 1062 to consider a different subject sample image. In various embodiments, blocks 1062, 1064, and 1066 may be repeated for each sample image. After all sample images have been considered, there may be stored in the location 450 of the storage memory numerous candidate confidence threshold comparison contour records generally similar to the candidate confidence threshold comparison contour record 700 shown in FIG. 14 and there may be stored in the location 452 of the storage memory 404 numerous grouped candidate set identifier records generally similar to the grouped candidate set identifier record 720 as shown in FIG. 15.

Once all sample images have been considered, the analyzer processor 400 may be directed to block 1068. In various embodiments, considering and processing all of the sample images in this way prior to further analysis may facilitate parallel processing and/or improved efficiency. In some embodiments, the further analysis performed by blocks 1068-1076 may be started in parallel with the initial analysis, once the initial analysis performed by blocks 1062, 1064, and 1066 is complete for some subset of sample images, but before it's been completed for all sample images. In some embodiments, parallelizing the initial and the further analysis in this way may facilitate making the processing even faster and/or more efficient (e.g., from the point of view of memory usage).

In various embodiments, blocks 1068-1076 may direct the analyzer processor 400 to identify and combine adjacent or overlapping contours as defined by the candidate confidence threshold comparison contour records stored in the location 450 of the storage memory 404. In various embodiments, additional or alternative ways of identifying and combining adjacent or overlapping contours as defined by the candidate confidence threshold comparison contour records stored in the location 450 of the storage memory 404 may be employed.

Referring to FIG. 26, block 1068 directs the analyzer processor 400 to consider a subject sample image. In various embodiments, block 1068 may direct the analyzer processor 400 to only consider a subject sample image that includes at least one contour defined by a candidate confidence threshold comparison contour record stored in the location 450 of the storage memory 404. In some embodiments, block 1068 may direct the analyzer processor 400 to start with a top left most sample image as a first subject sample image.

Block 1070 directs the analyzer processor 400 to consider an adjacent sample image. In various embodiments, block 1068 may direct the analyzer processor 400 to only consider an adjacent sample image that includes at least one contour defined by a candidate confidence threshold comparison contour record stored in the location 450 of the storage memory 404. In some embodiments, block 1068 may direct the analyzer processor 400 to only consider an adjacent sample image that has not been previously considered in combination with the subject image (for example in an instance where the current subject image was considered as an adjacent image).

Block 1072 directs the analyzer processor 400 to determine, for each of the candidate sets of adjacent sample image elements associated with the subject sample image, whether a sample image element of the candidate set of adjacent sample image elements associated with the subject sample image is adjacent to or overlapping (e.g., intersecting) with at least one sample image element of a candidate set of adjacent sample image elements associated with the adjacent sample image. In various embodiments, if the candidate sets of adjacent sample image elements are adjacent or overlapping, then they may be considered as a single candidate set of sample image elements that continues across more than one sample image.

In various embodiments, block 1072 may include code generally similar to that included in the block 646 of the flowchart 640 shown in FIG. 12.

If at block 1072, it is found that no sample image elements are adjacent or overlapping, then block 1072 directs the analyzer processor 400 to proceed to block 1076.

If at block 1072, it is found that there it is at least some overlapping or adjacent sample image elements, then the analyzer processor 400 is directed to block 1074 to join the overlapping sample image elements in a single candidate set of adjacent sample image elements.

Block 1074 directs the analyzer processor 400 to include the set of adjacent sample image elements associated with the adjacent sample image that were found to be adjacent or overlapping in the candidate set of adjacent sample image elements associated with the subject image. In various embodiments, block 1074 may include generally similar code to that included in the block 648 of the flowchart 640 shown in FIG. 12.

In some embodiments, block 1074 may direct the analyzer processor 400 to combine the contents of the grouped candidate set identifier records for the subject sample image and the adjacent sample image, such that a new grouped candidate set identifier record is stored in the location 452 of the storage memory 404. In some embodiments the updated or new grouped candidate set identifier record may be generally similar to the grouped candidate set identifier record 720 shown in FIG. 17.

In some embodiments, block 1074 may direct the analyzer processor 400 to delete or remove any former grouped candidate set identifier records that were copied or combined into the new grouped candidate set identifier record, such that redundant grouped candidate set identifier records are removed or deleted.

After block 1074 or if at block 1072 it is found that no sample image elements are adjacent or overlapping, the analyzer processor 400 proceeds to block 1076. Block 1076 directs the analyzer processor 400 to determine whether any adjacent sample images remain to be considered. If at block 1076, it is determined that there is at least one further sample image adjacent to the subject sample image that has not been considered, then block 1076 directs the analyzer processor 400 to return to block 1070 and consider a further adjacent sample image and blocks 1070-1074 may be executed.

If at block 1076 it is determined that all sample images adjacent to the subject sample image have been considered, then block 1076 directs the analyzer processor 400 to return to block 1068 and a new subject sample image is considered.

In various embodiments, execution of blocks 1068-1076 may be repeated until all subject sample images or all possible sample image pairs or groups have been considered.

In various embodiments, the block of codes 506 of the flowchart 500 shown in FIG. 6 may include code for directing the analyzer processor 400 to combine any grouped candidate set identifier records that include a common contour into a single grouped candidate set identifier record.

While sample image elements described herein in one embodiment are pixels, in various embodiments, further or alternative types of sample image elements, which may be identifiable as objects or instances or parts of a studied feature, may be used. For example, in some embodiments sample image elements may include groups of pixels. In some embodiments, a sample image may be broken down into a matrix or grid of groups of pixels, each group being a set of adjacent pixels such as a 10 pixel×10 pixel square, for example, which may be referred to herein as a pixel square or pixel group and may act as a sample image element.

In some embodiments, where pixel groups are used as sample image elements, wherever pixel values are described as considered herein, a representative value for a pixel group may be used. For example, in some embodiments, where pixel groups are used as sample image elements, a block generally similar to the block 504 shown in FIG. 6 may direct the analyzer processor 400 to determine an average pixel value for each pixel group and to use the average pixel values as inputs for the one or more functions. In some embodiments, a median value may be used instead of the average value. In some embodiments, a maximum value may be used instead of the average value. In some embodiments, using the maximum value may be useful in the context of immunofluorescence images, for example. In various embodiments, using groups of pixels as sample image elements instead of individual pixels may help reduce processing requirements for analyzing the sample images. In some embodiments, using groups of pixels as sample image elements instead of individual pixels may help focus on large-scale features and/or context, rather than tiny details that may be relatively less important from the point of view of the task at hand. In various embodiments, focusing on large-scale features and/or context like this may improve specificity and/or sensitivity of the analysis.

For example, in some embodiments, a sample image may be downsized from 2000×2000 to 1000×1000 using pixel squares. In some embodiments, this may be preferable, for example, in case the highest possible resolution is not needed and in such embodiments, a 1000×1000 sample image of pixel squares may be input into a property identifying neural network, such as a tumor detecting and/or grading neural network. In some embodiments, pixel squares may be used to downsize 20000×20000 pixels in original image coordinates to 2000×2000 pixels input to a tissue detecting neural network acting as an undesirable image element detecting neural network having a limited field of view of 31×31 pixels, for example.

In some embodiments, the pixel group size may be greater than one pixel or less than one pixel. In such embodiments, use of pixel groups may effectively resize the original image, which may be done using standard image resizing methods (e.g., bilinear or bicubic interpolation).

In some embodiments, pixel groups may be used in the undesirable image element detecting neural network, with a pixel group size larger than what may be used for the property identifying neural network. In some embodiments, using pixel groups may be an effective way to make the undesirable image element detecting neural network see even larger areas (in terms of square micrometers) at a time.

In some embodiments, the image analyzer 12 may be configured to use the undesirable image element detecting neural network as a filter such that if a pixel is deemed undesirable, then any findings, such as, by the property identifying neural network, from that pixel are ignored (e.g., no cancer is to be detected outside of tissue).

In some embodiments, the sample image elements may include results (outputs) of image processing and/or filtering functions.

In some embodiments, the image analyzer 12 may be configured to cause an alternative or additional function to be applied to the plurality of sample images to determine the plurality of confidence scores. For example, in some embodiments, in case of immunofluorescence images, the function may use a signal intensity itself as the confidence score. In some embodiments, the function may include a function that picks a single channel from many, for example.

In various embodiments, the at least one representative confidence score may be determined in additional or alternative ways. For example, in some embodiments, block 508 of the flowchart 500 may direct the analyzer processor 400 to determine a representative confidence score by applying one or more additional or alternative functions to the sample image elements. In some embodiments, for example, block 508 may direct the analyzer processor 400 to apply a different neural network, such as, for example, a confirmation neural network, to the sample image elements to determine the representative confidence score. In some embodiments, for example, the property identifying neural network may have a relatively smaller limited field of view, and it may be trained to accurately render the contours of any regions that may generally look like tumor, for example, whereas the confirmation neural network may have a relatively larger limited field of view and it may be trained to generally classify the tumor regions, for example, rather than being able to render their contours with high accuracy. In some embodiments, using two different neural networks like this may help improve the specificity and/or sensitivity of the overall analysis.

In some embodiments, the representative high confidence score may be identified in alternative or additional ways. For example, in some embodiments, blocks 842 and 844 may direct the analyzer processor 400 to determine an Nth highest confidence score and to use the Nth highest confidence score as the representative high confidence score, wherein N is a number greater than 1. In some embodiments N may be 3, for example. In various embodiments, using the Nth highest confidence score, wherein N is greater than 1 may facilitate avoiding or reducing the likelihood that a single outlier or noisy pixel value may cause a false confirmation.

In various embodiments, the property identifying neural network may use a limited field of view window surrounding a central pixel to provide context in determining a confidence score for the central pixel. In various embodiments, to have the central pixel not be ambiguous (and also for other reasons), the field of view window may have odd numbered dimensions, e.g. 265×265 pixels.

In some embodiments, a limited field of view window dimension for a property identifying neural network may be 2^{N}−1 for some N that is a natural number (e.g., N=8 resulting in a field of view window dimension of 255).

In some embodiments, a simpler more hard-coded analysis may be used to determine whether a candidate sample image is an undesirable sample image at block 544. For example, in some embodiments, block 544 may direct the analyzer processor 400 to determine whether each sample image defined by the candidate sample image definition record 580 shown in FIG. 9 is essentially blank or white and thus does not include any depiction of tissue.

In some embodiments identifying undesirable sample image elements may provide a filter such that if a sample image element is deemed undesirable, then any findings from the area of the pathology image 102 corresponding to that part of the sample image are ignored (e.g., no cancer is to be detected outside of tissue). For example, in some embodiments, block 506 of the flowchart 500 may include a block of codes for directing the analyzer processor 400 to update the binary images stored in the location 450 of the storage memory 404 such that each pixel position associated with a pixel identified as an undesirable sample image element is set to 0.

While specific embodiments of the present disclosure have been described and illustrated, such embodiments should be considered illustrative of the present disclosure only and not as limiting the present disclosure as construed in accordance with the accompanying claims.

What is claimed is:

1. A method of identifying sets of image elements as representative of a sample property for pathology, the method comprising:
receiving pathology image data representing a plurality of sample images representing respective adjacent or overlapping portions of a sample for analysis in pathology, each of the plurality of sample images including a plurality of sample image elements;
causing one or more functions to be applied to the plurality of sample images to determine a plurality of confidence scores, each of the plurality of confidence scores associated with one of the plurality of sample image elements and representing a level of confidence that the associated sample image element represents the sample property;
comparing at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold;
determining whether at least one representative confidence score associated with the candidate set of adjacent sample image elements is greater than a confirmation confidence threshold; and
when the at least one representative confidence score is greater than the confirmation confidence threshold, associating the candidate set of adjacent sample image elements with a sample property identifier for identifying the candidate set of adjacent sample image elements as representing the sample property.

2. The method of claim 1 comprising determining the at least one representative confidence score based on the confidence scores associated with the candidate set of adjacent sample image elements.

3. The method of claim 2 wherein determining the at least one representative confidence score comprises identifying the at least one representative confidence score from the confidence scores associated with the candidate set of adjacent sample image elements.

4. The method of claim 1 wherein the confirmation confidence threshold is greater than the candidate confidence threshold.

5. The method of claim 1 wherein the plurality of sample images includes a first sample image and a second sample image and wherein comparing at least one of the plurality of confidence scores with the candidate confidence threshold to identify the candidate set of adjacent sample image elements comprises:
comparing confidence scores associated with sample image elements included in the first sample image with the candidate confidence threshold to identify a first sample image set of adjacent sample image elements, each sample image element of the first sample image set of adjacent sample image elements associated with a confidence score greater than the candidate confidence threshold;
including the first sample image set of adjacent sample image elements in the candidate set of adjacent sample image elements;
comparing confidence scores associated with sample image elements included in the second sample image with the candidate confidence threshold to identify a second sample image set of adjacent sample image elements, each sample image element of the second sample image set of adjacent sample image elements associated with a confidence score greater than the candidate confidence threshold;
determining whether at least one sample image element of the first sample image set is adjacent to or overlapping with at least one sample image element of the second sample image set; and
in response to determining that at least one sample image element of the first sample image set is adjacent to or overlapping with at least one sample image element of the second sample image set, including the second sample image set of adjacent sample image elements in the candidate set of adjacent sample image elements.

6. The method of claim 5 wherein the at least one representative confidence score includes a representative high confidence score and determining whether the at least one representative confidence score is greater than the confirmation confidence threshold comprises:
determining the representative high confidence score based on the confidence scores associated with the candidate set of adjacent sample image elements; and
determining whether the representative high confidence score is greater than the confirmation confidence threshold.

7. The method of claim 6 wherein determining the representative high confidence score comprises:
determining a first candidate representative high confidence score associated with a sample image element of the first sample image set;
determining a second candidate representative high confidence score associated with a sample image element of the second sample image set; and
determining the representative high confidence score as the greatest of the first and second candidate representative high confidence scores.

8. The method of claim 1 wherein associating the candidate set of adjacent sample image elements with the sample property identifier comprises producing signals for causing the candidate set of adjacent sample image elements to be displayed in association with the sample property.

9. The method of claim 1 comprising determining the plurality of sample images based on the received pathology image data.

10. The method of claim 9 wherein determining the plurality of sample images based on the received pathology image data comprises:
determining a candidate set of sample images;
identifying at least one undesirable sample image of the candidate set of sample images; and
determining the plurality of sample images as a subset of the candidate set of sample images, the plurality of sample images not including the at least one undesirable sample image.

11. The method of claim 10 wherein identifying the at least one undesirable sample image comprises determining that the at least one undesirable sample image generally lacks depiction of tissue.

12. The method of claim 10 wherein identifying the at least one undesirable sample image comprises causing sample image elements of the candidate set of sample images to be input into an undesirable image element detecting neural network.

13. The method of 9 wherein the pathology image data includes a representation of a single pathology image and wherein determining the plurality of sample images based on the received pathology image data comprises determining the plurality of sample images as portions of the pathology image.

14. The method of claim 9 wherein determining the plurality of sample images from the pathology image data comprises, for each of the plurality of sample images:
determining a position of the sample image within the pathology image and associating the position with the sample image; and
associating a width and a height for the sample image with the sample image.

15. The method of claim 1 wherein the plurality of sample images are overlapping by an overlap width and an overlap height between adjacent sample images.

16. The method of claim 1 wherein causing the one or more functions to be applied to the plurality of sample images to determine a plurality of confidence scores comprises causing each of the plurality of sample images to be input into a property identifying neural network.

17. The method of claim 1 wherein:
causing the one or more functions to be applied to the plurality of sample images to determine a plurality of confidence scores comprises causing each of the plurality of sample images to be input into a property identifying neural network, the property identifying neural network having a limited field of view having a field of view width and a field of view height;
the plurality of sample images are overlapping by an overlap width and an overlap height between adjacent sample images; and
the overlap height is greater than or equal to the field of view height minus one image element height and the overlap width is greater than or equal to the field of view width minus one image element width.

18. The method of claim 17 wherein the overlap width is equal to the field of view width minus one image element width and the overlap height is equal to the field of view height minus one image element height.

19. The method of claim 1 wherein the candidate set of adjacent sample image elements includes sample image elements from more than one of the plurality of sample images.

20. The method of claim 1 wherein the sample property includes at least one of:
a biomarker;
a type of tissue;
epithelial tissue;
stroma;
neither epithelial tissue nor stroma;
properties of a cell;
nuclei;
cell membrane;
mitotic status;
cells expressing a specific protein;
cells expressing Ki-67;
a cell or group of cells having a condition;
a cancer cell;
a group of cancer cells forming a tumor;
an immune cell;
a cell having a pathological condition;
a necrotic cell;
a histologic pattern;
a Gleason pattern; or
a tumor grade.

21. The method of claim 1 wherein the sample image element is a pixel.

22. A system for identifying sets of image elements as representative of a sample property for pathology, the system comprising:
a memory that stores instructions; and
at least one processor configured to execute the instructions to perform operations including:
receiving pathology image data representing a plurality of sample images representing respective adjacent or overlapping portions of a sample for analysis in pathology, each of the plurality of sample images including a plurality of sample image elements;
causing one or more functions to be applied to the plurality of sample images to determine a plurality of confidence scores, each of the plurality of confidence scores associated with one of the plurality of sample image elements and representing a level of confidence that the associated sample image element represents the sample property;
comparing at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold;
determining whether at least one representative confidence score associated with the candidate set of adjacent sample image elements is greater than a confirmation confidence threshold; and
when the at least one representative confidence score is greater than the confirmation confidence threshold, associating the candidate set of adjacent sample image elements with a sample property identifier for identifying the candidate set of adjacent sample image elements as representing the sample property.

23. A non-transitory computer-readable medium having stored thereon instructions that when executed by at least one processor cause the at least one processor to perform operations including:
receiving pathology image data representing a plurality of sample images representing respective adjacent or overlapping portions of a sample for analysis in pathology, each of the plurality of sample images including a plurality of sample image elements;
causing one or more functions to be applied to the plurality of sample images to determine a plurality of confidence scores, each of the plurality of confidence scores associated with one of the plurality of sample image elements and representing a level of confidence that the associated sample image element represents the sample property;
comparing at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold;
determining whether at least one representative confidence score associated with the candidate set of adjacent sample image elements is greater than a confirmation confidence threshold; and
when the at least one representative confidence score is greater than the confirmation confidence threshold, associating the candidate set of adjacent sample image elements with a sample property identifier for identifying the candidate set of adjacent sample image elements as representing the sample property.

24. A system for identifying sets of image elements as representative of a sample property for pathology, the system comprising:
means for receiving pathology image data representing a plurality of sample images representing respective adjacent or overlapping portions of a sample for analysis in pathology, each of the plurality of sample images including a plurality of sample image elements;
means for causing one or more functions to be applied to the plurality of sample images to determine a plurality of confidence scores, each of the plurality of confidence scores associated with one of the plurality of sample image elements and representing a level of confidence that the associated sample image element represents the sample property;
means for comparing at least one of the plurality of confidence scores with a candidate confidence threshold to identify a candidate set of adjacent sample image elements of the plurality of sample image elements, each sample image element of the candidate set of adjacent sample image elements associated with a confidence score greater than the candidate confidence threshold;
means for determining whether at least one representative confidence score associated with the candidate set of adjacent sample image elements is greater than a confirmation confidence threshold; and
means for, when the at least one representative confidence score is greater than the confirmation confidence threshold, associating the candidate set of adjacent sample image elements with a sample property identifier for identifying the candidate set of adjacent sample image elements as representing the sample property.
